(19) United States
(12) Patent Application Publication
(10) Pub. No.: US 2021/0060121 A1
(43) Pub. Date: Mar. 4, 2021

(54) NOVEL ANTIBIOTICS AND METHODS OF USING THEM

(71) Applicants: VERSITECH LIMITED, Hong Kong (CN); THE HONG KONG POLYTECHNIC UNIVERSITY, Hong Kong (CN)

(72) Inventors: Xuechen LI, Hong Kong (CN); Kang JIN, Hong Kong (CN); Sheng CHEN, Hong Kong (CN); Kathy Hiu Laam PO, Hong Kong (CN)

(21) Appl. No.: 16/958,028

(22) PCT Filed: Dec. 20, 2018
(86) PCT No.: PCT/CN2018/122374

§ 371 Date: Jun. 25, 2020

Related U.S. Application Data
(60) Provisional application No. 62/613,292, filed on Jan. 3, 2018.

(51) Int. Cl. A61K 38/15

(52) U.S. Cl. CPC A61K 38/15

(57) ABSTRACT

The invention pertains to a new class of cyclic peptides as antibiotics. The antibiotics of the invention are derivatives of teixobactin. Teixobactin is modified to develop teixobactin analogues with improved antibacterial and therapeutic properties. Pharmaceutical compositions comprising the antibiotics of the invention and pharmaceutically acceptable carriers or excipients are also provided. Further provided are methods of treating an infection by an agent in a subject by administering to the subject the antibiotics described herein.

### BACKGROUND OF THE INVENTION

The emergence of multidrug resistance in bacterial pathogens has severely challenged public health management. Increasing cases of antibiotic-resistant infections worldwide cost billions in health care costs and productivity losses. Despite substantial research, reliable solutions to address the rapid development of resistance to clinically important antimicrobial agents remain elusive. Further, the current drug development programs are insufficient to provide therapeutic coverage in the foreseeable future, partly due to scientific challenges in discovering new classes of antibiotics, and new compounds in established classes, as well as financial considerations of pharmaceutical companies. Therefore, global dissemination of antibiotic resistant organisms remains unchecked.

Teixobactin was discovered recently from Eleftheria terrae, a gram-negative bacterium found in soil. Teixobactin is a new broad-spectrum antibiotic that can act by blocking the formation of the cell wall against a wide range of gram-positive bacteria.

### BRIEF SUMMARY OF THE INVENTION

Certain embodiments of the invention relate to a new class of cyclic peptides as antibiotics. The antibiotics of the invention are derivatives of teixobactin. Teixobactin has a unique structural motif, as shown in FIG. 1. The structural motif of teixobactin is modified to develop teixobactin analogues, which represent the antibiotics of the invention. The antibiotics of the invention exhibit antibacterial and therapeutic properties superior to those of teixobactin. The novel antibiotics described herein offer substantial promise to combat the serious problem of antibiotic-resistant bacteria.

In specific embodiments, the antibiotics of the invention have a structure of Formula I, I′ II, II′, III, III′, IV, IV′, V, V′ or VI, VI′ provided below:

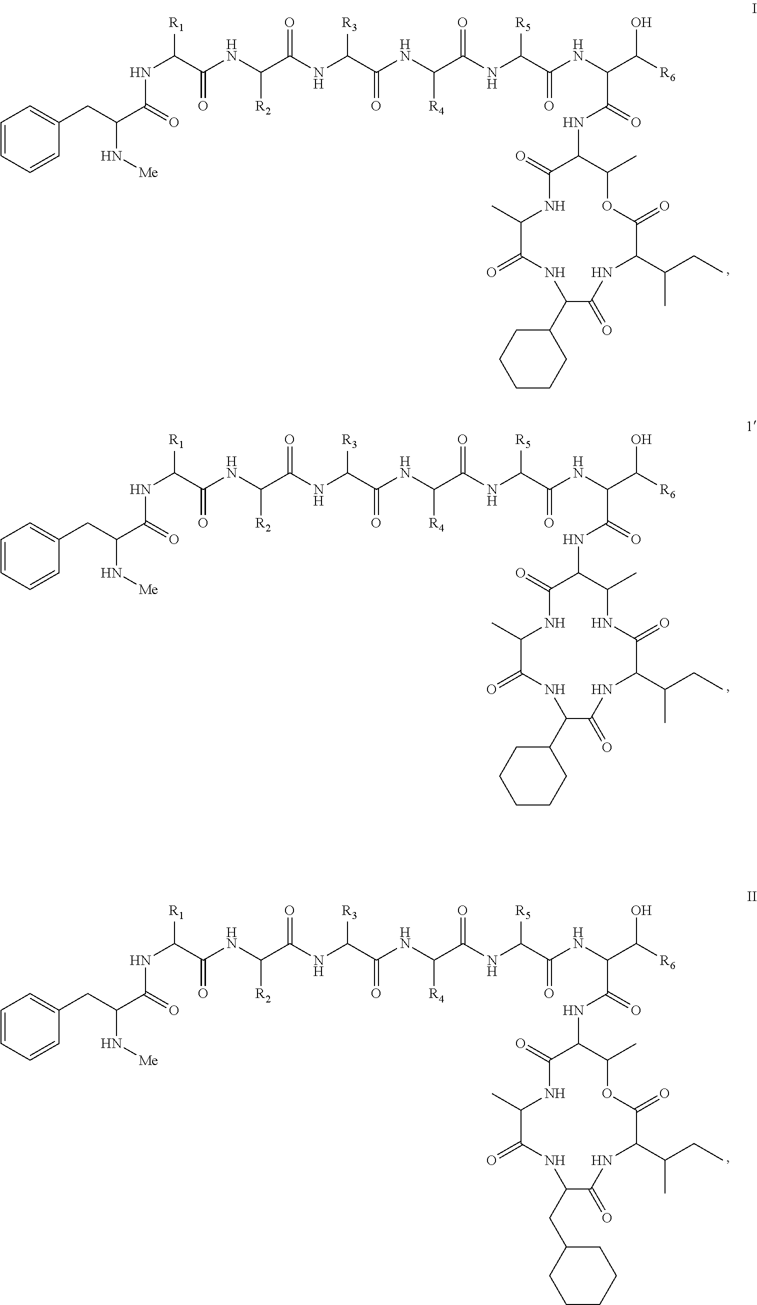

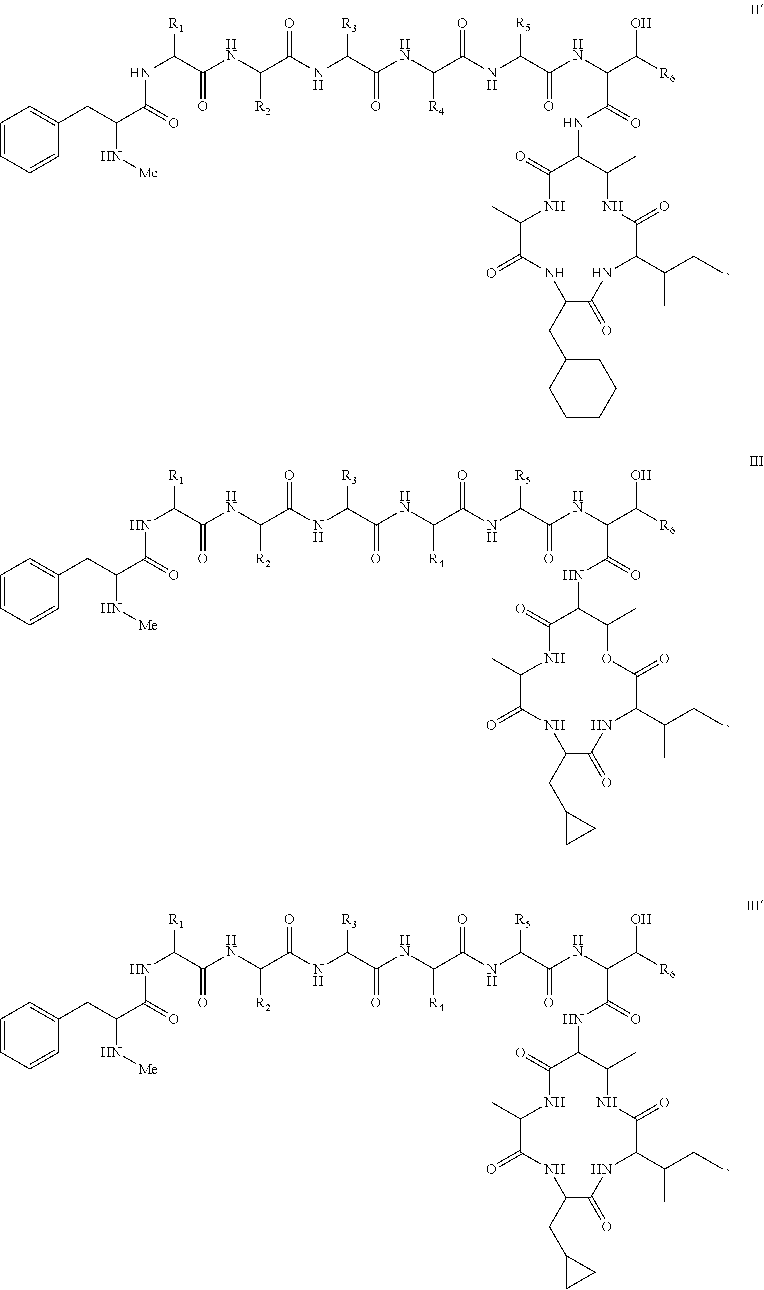

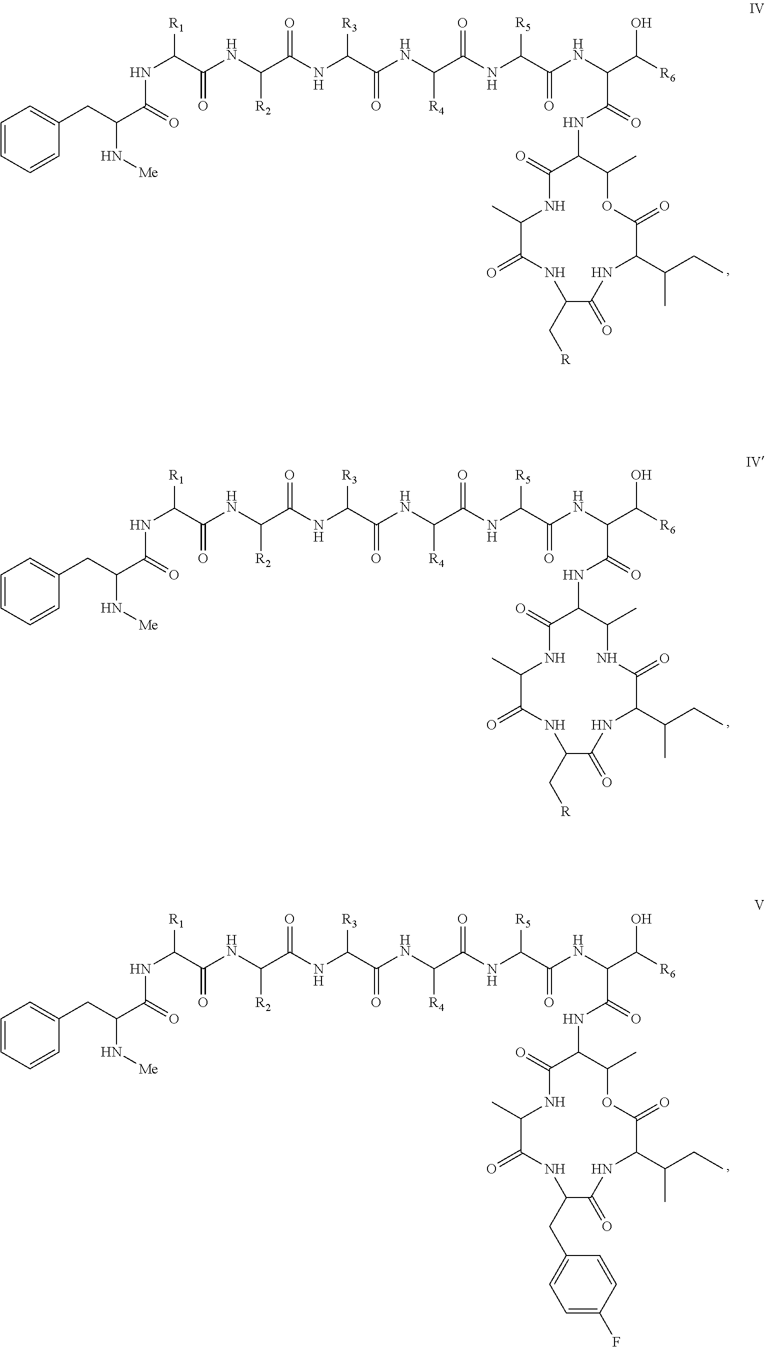

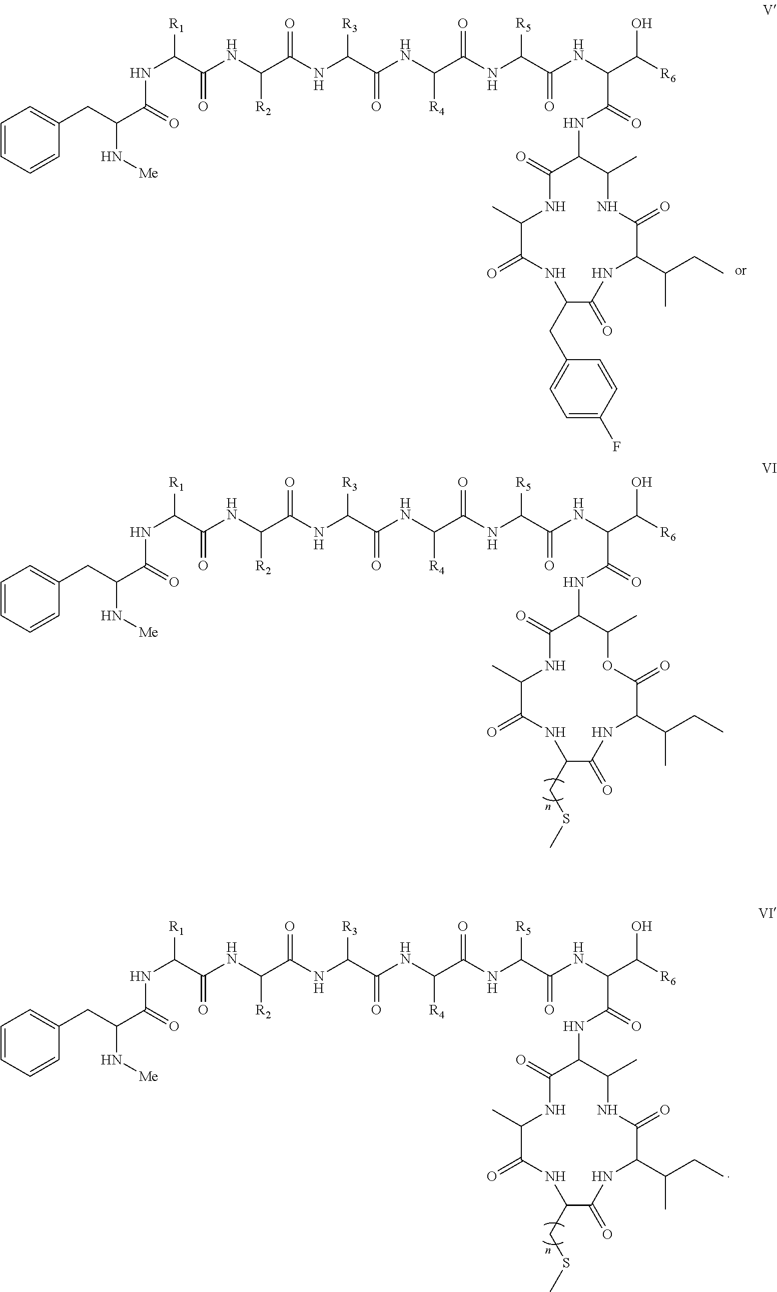

In preferred embodiments, each of R₁ to R₅ is a side chain of an amino acid, for example, a side chain of leucine, isoleucine, glutamine, serine, threonine, D-allo isoleucine, lysine, or arginine. In certain embodiments, R₆ is H or a C₁ to C₉ linear or branched alkyl group, preferably, methyl. R group of Formula IV or IV′ can be a C₂-C₈ linear or branched alkyl chain.

Pharmaceutical compositions comprising the antibiotics of the invention and pharmaceutically acceptable carriers or excipients are also provided. Further provided are methods of treating an infection by an agent in a subject by administering to the subject the antibiotics described herein.

### BRIEF DESCRIPTION OF THE DRAWINGS

FIG. 1. Structure of teixobactin.

FIG. 2. In vivo antibacterial activities of certain antibiotics of the invention. Efficacy of teixobactin and certain antibiotics of the invention for treating infections caused by Staphylococcus aureus strain ATCC43300 was studied in wax worm larvae (Galleria mellonella) model. PBS, control group without infection and treated with PBS: All other groups (20 each group) were infected with 10⁷ CFU of S. aureus strain ATCC43300 followed by no treatment or treatment with one dose of vancomycin at 10 mg/kg (A-10) or 50 mg/kg (A-50), teixobactin at 10 mg/kg (B-10) or 50 mg/kg (B-50), or one dose of the representative analogue, Chg10-teixobactin, at 10 mg/kg (C-10) or 50 mg/kg (C-50).

FIG. 3. Structure and LC-MS spectra of Chg10-teixobactin. UV trace and corresponding mass from LC-MS analysis of compound Chg10-teixobactin. ESI: calculated for C₆₀H₉₉N₁₂O₁₅⁺[M+H+]: 1227.7; found: 1227.8.

FIG. 4. Structure and LC-MS spectra of Met10-teixobactin/Methibactin. White solid, 2.9 mg, Yield 44% (the final step). UV trace and corresponding mass from LC-MS analysis of compound T61 ESI: calculated for C₅₇H₉₅N₁₂O₁₅S⁺[M+H⁺]: 1219.7; found: 1219.7.

FIG. 5. Structure and LC-MS spectra of compound L-Phe(4-F)10-teixobactin. White solid, 2.9 mg, yield 45% (the final step). UV trace and corresponding mass from LC-MS analysis of compound L-Phe(4-F)10-teixobactin. Gradient: 5-95% CH₃CN/H₂O with 0.1% TFA over 15 min at a flow rate of 0.6 mL/min. ESI: calculated for C₆₁H₉₄FN₁₂O₁₅⁺[M+H⁺]: 1253.7; found: 1253.8

FIG. 6. Structure and LC-MS spectra of compound Nle10-teixobactin. White solid, 2.9 mg, yield 45% (the final step). UV trace and corresponding mass from LC-MS analysis of compound Nle10-teixobactin. Gradient: 5-95% CH₃CN/H₂O with 0.1% TFA over 15 min at a flow rate of 0.6 mL/min. ESI: calculated for C₅₈H₉₇N₁₂O₁₅⁺[M+H⁺]: 1201.7; found: 1201.8

FIG. 7. Structure and LC-MS spectra of compound L-Nva10-teixobactin. White solid, 3.1 mg, yield 48% (the final step). UV trace and corresponding mass from LC-MS analysis of compound L-Nva10-teixobactin. Gradient: 5-95% CH₃CN/H₂O with 0.1% TFA over 15 min at a flow rate of 0.6 mL/min. ESI: calculated for C₅₇H₉₅N₁₂O₁₅⁺[M+H⁺]: 1187.7; found: 1187.7

FIG. 8. Structure and LC-MS spectra of compound L-Cha10-teixobactin. White solid, 3.3 mg, yield 50% (the final step). UV trace and corresponding mass from LC-MS analysis of compound L-Cha10-teixobactin. Gradient: 5-95% CH₃CN/H₂O with 0.1% TFA over 15 min at a flow rate of 0.6 mL/min. ESI: calculated for C₆₁H₁₀₁N₁₂O₁₅⁺[M+H⁺]: 1241.7; found: 1241.9

FIG. 9. Structure and LC-MS spectra of compound L-β-cyclopropyl-Ala10-Teixobactin. White solid, 2.8 mg, Yield 43% (the final step). UV trace and corresponding mass from LC-MS analysis of compound L-β-cyclopropyl-Ala10-Teixobactin. Gradient: 5-95% CH₃CN/H₂O with 0.1% TFA over 15 min at a flow rate of 0.6 mL/min. ESI: calculated for C₅₈H₉₅N₁₂O₁₅⁺[M+H⁺]: 1199.7; found: 1199.9

FIG. 10. Structure and LC-MS spectra of compound T83. UV trace and corresponding masses from LC-MS analysis of compound T83. Gradient: 5-95% CH3CN/H2O with 0.1% TFA over 15 min at a flow rate of 0.6 mL/min. ESI: calculated for C60H100N13O14+[M+H+]: 1226.7; found: 1226.9

FIG. 11. Structure and LC-MS spectra of compound T84. Gradient: 5-95% CH3CN/H20 with 0.1% TFA over 15 min at a flow rate of 0.6 mL/min. ESI: calculated for C58H98N13O14+[M+H+]: 1200.7; found: 1200.8

FIG. 12. Structure and LC-MS spectra of compound of compound T85. Gradient: 5-95% CH₃CN/H₂O with 0.1% TFA over 15 min at a flow rate of 0.6 mL/min. ESI: calculated for C₅₇H₉₆N₁₃O₁₄⁺[M+H⁺]: 1186.7; found: 1186.7

FIG. 13. Structure and LC-MS spectra of compound of compound T86. Gradient: 5-95% CH₃CN/H₂O with 0.1% TFA over 15 min at a flow rate of 0.6 mL/min. ESI: calculated for C₅₇H₉₆N₁₃O₁₄S⁺[M+H⁺]: 1218.7; found: 1218.8

FIG. 14. Structure and LC-MS spectra of compound of compound T87. Gradient: 5-95% CH₃CN/H₂O with 0.1% TFA over 5 min at a flow rate of 0.4 mL/min. ESI: calculated for C₆₃H₁₀₆N₁₃O₁₄⁺[M+H⁺]: 1297.5; found: 1297.3

FIG. 15. Structure and LC-MS spectra of compound of compound T88. Gradient: 5-95% CH₃CN/H₂O with 0.1% TFA over 5 min at a flow rate of 0.4 mL/min. ESI: calculated for C₆₃H₁₀₅N₁₂O₁₄⁺[M+H⁺]: 1253.8; found: 1254.2.

### DETAILED DISCLOSURE OF THE INVENTION

As used herein, the singular forms “a,” “an” and “the” are intended to include the plural forms as well, unless the context clearly indicates otherwise. Further, to the extent that the terms “including,” “includes,” “having,” “has,” “with,” or variants thereof are used in either the detailed description and/or the claims, such terms are intended to be inclusive in a manner similar to the term “comprising.”

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which will depend in part on how the value is measured or determined, i.e., the limitations of the measurement system. Where particular values are described in the application and claims, unless otherwise stated the term “about” meaning within an acceptable error range for the particular value should be assumed. In the context of compositions containing amounts of ingredients where the terms “about” or “approximately” are used, these compositions contain the stated amount of the ingredient with a variation (error range) of 0-10% around the value (X±10%).

In the present disclosure, ranges are stated in shorthand, so as to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values.

When ranges are used herein, such as for dose ranges, combinations and subcombinations of ranges (e.g., subranges within the disclosed range), specific embodiments therein are intended to be explicitly included.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of an antibiotic of the invention that is pharmaceutically acceptable and that possesses the desired antibacterial activity of the parent antibiotic. In particular, such salts are non-toxic may be inorganic or organic acid addition salts and base addition salts.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

“Treating” or “treatment” of any disease or disorder refers, in one embodiment, to ameliorating the infection (i.e., arresting or reducing the development of the infection or at least one of the clinical symptoms thereof). “Treating” or “treatment” includes ameliorating at least one physical parameter, which may not be discernible by the subject. “Treating” or “treatment” also includes modulating the infection, either physically, (e.g., stabilization of a discernible symptom), physiologically, (e.g., stabilization of a physical parameter), or both. “Treating” or “treatment” further includes delaying the onset of the infection.

As used herein, the terms “reducing,” “inhibiting,” “blocking,” “preventing,” “alleviating,” or “relieving” when referring to an antibiotic, mean that the antibiotic brings down the occurrence, severity, size, volume, or associated symptoms of an infection, event, or activity by at least about 7.5%, 10%, 12.5%, 15%, 17.5%, 20%, 22.5%, 25%, 27.5%, 30%, 35%, 40%, 45%, 50%, 55%, 60%, 65%, 70%, 75%, 80%, 90%, or 100% compared to how the infection would normally exist without application of the antibiotic or a composition comprising the antibiotic. The terms “increasing,” “elevating,” “enhancing,” “upregulating,” “improving,” or “activating” when referring to an antibiotic mean that the antibiotic increases the occurrence of a condition, event, or activity by at least about 7.5%, 10%, 12.5%, 15%, 17.5%, 20%, 22.5%, 25%, 27.5%, 30%, 35%, 40%, 45%, 50%, 55%, 60%, 65%, 70%, 75%, 80%, 90%, 100%, 150%, 200%, 250%, 300%, 400%, 500%, 750%, or 1000% compared to how the infection would normally exist without application of the antibiotic or a composition comprising the antibiotic.

The term “effective amount” or “therapeutically effective amount” refers to that amount of an antibiotic described herein that is sufficient to effect the intended application including but not limited to infection treatment. The therapeutically effective amount may vary depending upon the subject and infection being treated, e.g., the weight and age of the subject, the severity of the infection, the manner of administration and the like, which can readily be determined by one of ordinary skill in the art. The term also applies to a dose that will induce a particular response in vitro, e.g., reduction of proliferation killing of target bacterium. The specific dose will vary depending on the particular compounds chosen, the dosing regimen to be followed, whether it is administered in combination with other compounds, timing of administration, the tissue to which it is administered, and the physical delivery system in which it is carried.

A “sub-therapeutic amount” of an agent is an amount less than the effective amount for that agent, but which when combined with an effective or sub-therapeutic amount of another agent or therapy can produce a desired result, due to, for example, synergy in the resulting therapeutic benefit for the patient, or reduced side effects associated with the antibiotics administered to the patient.

A “synergistically effective” therapeutic amount or “synergistically effective” amount of an agent is an amount which, when combined with an effective or subtherapeutic amount of another agent or therapy, produces a greater effect than when either of the two agents are used alone. A synergistically effective therapeutic amount of an agent produces a greater effect when used in combination than the additive effects of each of the two agents or therapies when used alone. The term “greater effect” encompasses not only a reduction in symptoms of the disorder to be treated, but also an improved side effect profile, improved tolerability, improved patient compliance, improved efficacy, or any other improved clinical outcome.

The terms “co-administration,” “administered in combination with,” and their grammatical equivalents encompass administration of two or more agents to a subject so that both agents and/or their metabolites are present in the subject at the same time. Co-administration includes simultaneous administration in separate compositions, administration at different times in separate compositions, or administration in a composition in which both agents are present. Co-administered agents may be in the same formulation. Co-administered agents may also be in different formulations.

“Subject” refers to an animal, such as a mammal, for example a human. The methods described herein can be useful in both pre-clinical human therapeutics and veterinary applications. In some embodiments, the subject is a mammal (such as an animal model of disease), and in some embodiments, the subject is human.

The terms “simultaneous” or “simultaneously” as applied to administering agents to a subject refer to administering one or more agents at the same time, or at two different time points that are separated by no more than 1 hour. The term “sequentially” refers to administering more than one agent at two different time points that are separated by more than 1 hour, e.g., about 2 hours, about 5 hours, 8 hours, 1 day, 2 days, 3 days, 4 days, 5 days, 6 days, 7 days or even longer.

Teixobactin exhibits promising activities against an array of bacterial pathogens. To date, teixobactin was only obtained by isolating from natural sources, partly because is difficult, if not impossible, to generate teixobactin analogues with structural diversities. For example, Teixobactin contains a (2S, 4S) enduracididine (L-allo-End), which is not commercially available and is difficult to synthesize. Therefore, generating L-allo-End containing teixobactin analogues via chemical synthesis is also difficult.

The invention discloses that L-allo-End residues could be replaced with hydrophobic, unnatural amino acid residues including norleucine (Nle), norvaline (Nva), cyclohexylglycine (Chg), cyclohexylalanine (Cha), cyclopropyl-Ala, Abu (aminobutyric acid), Aib (2-Aminoisobutyric acid), S-(tert-Butylthio)-L-cysteine (Cys(StBu)), etc.

Accordingly, the instant invention discloses synthetic class of teixobactin derivatives having desirable antibacterial activities. The antibiotics of the present invention can be chemically synthesized. The antibiotics of the invention are active against Gram-positive bacteria, including methicillin-resistant Staphylococcus aureus (MRSA), vancomycin-resistant Enterococcus (VRE) and Mycobacterium tuberculosis via synthetic tailoring of teixobactin core structure.

In certain embodiments, the antibiotics of the invention have Formula I or I′:

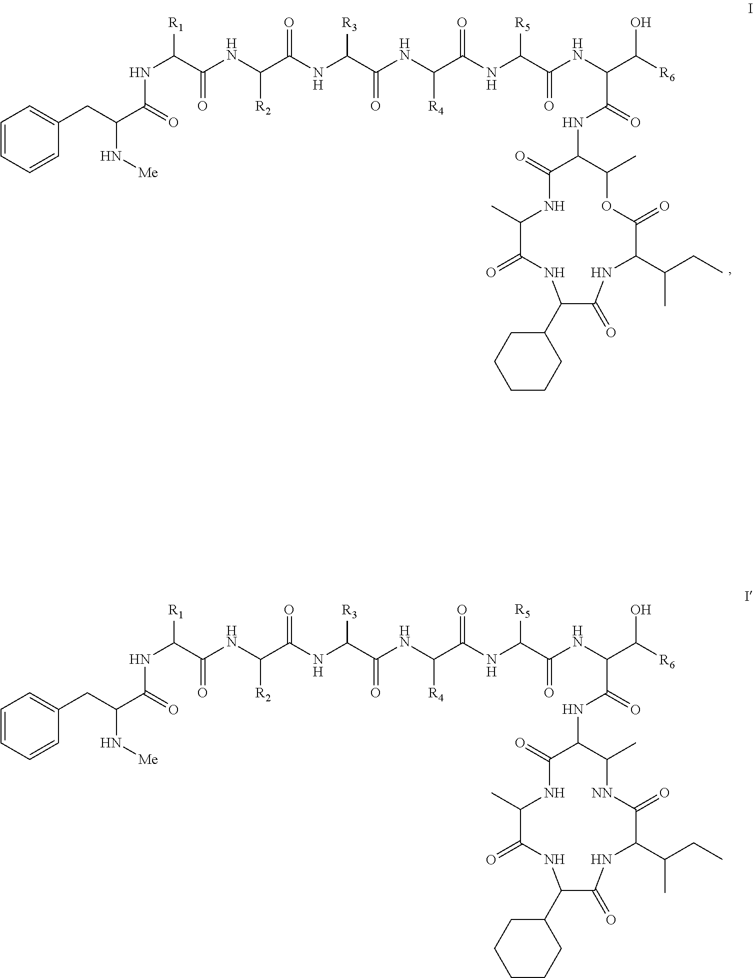

In some antibiotics of Formula I or I′, each of R₁ to R₅ is a side chain of amino acids, including natural or unnatural amino acids. In preferred embodiments of Formula I or I′, each of R₁ to R₅ is independently a side chain of leucine, isoleucine, glutamine, serine, threonine, D-allo isoleucine, lysine, or arginine. In specific embodiments of the antibiotics of Formula I or I′: R₁ is the side chain of isoleucine, R₂ is the side chain of serine or threonine, R₃ is the side chain of glutamine, R₄ is the side chain of isoleucine or D-allo isoleucine and/or R₅ is the side chain of isoleucine or D-allo isoleucine. In additional antibiotics of formula I, R₆ is H or a C₁ to C₉ linear or branched alkyl group, preferably, methyl.

In some embodiments, the antibiotics of the invention have Formula II or II′:

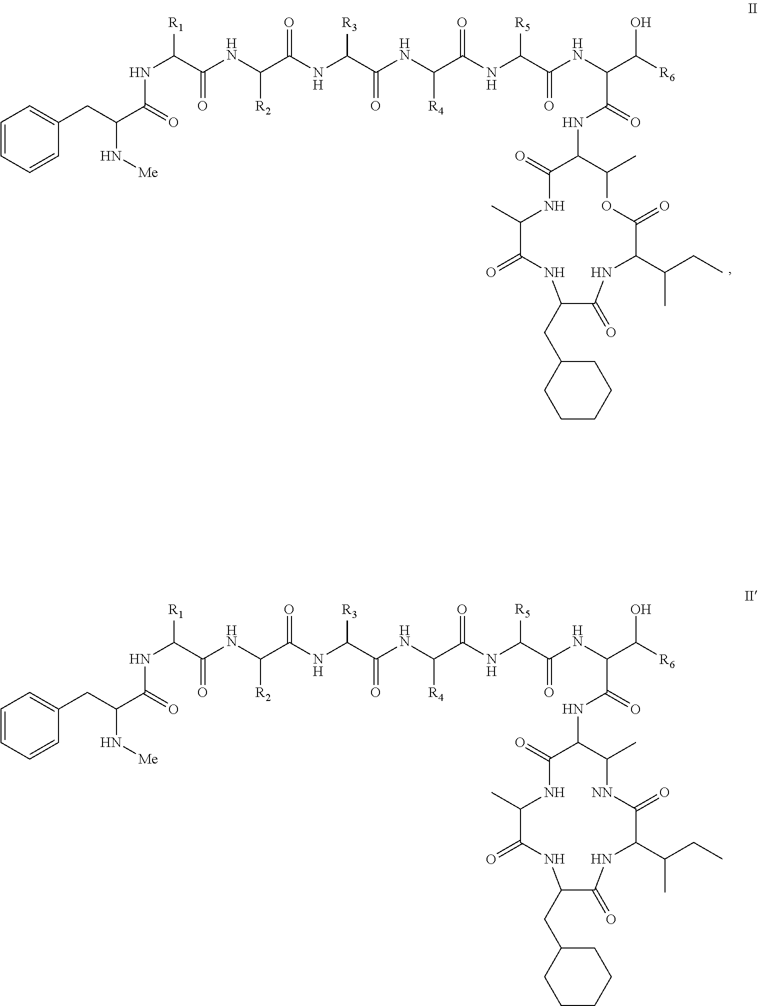

In some antibiotics of Formula II or II′, each of R₁ to R₅ is a side chain of amino acids, including natural or unnatural amino acids. In preferred embodiments of Formula II or II′, each of R₁ to R₅ is independently a side chain of leucine, isoleucine, glutamine, serine, threonine, D-allo isoleucine, lysine, or arginine. In specific embodiments of the antibiotics of Formula II or II′: R₁ is the side chain of isoleucine, R₂ is the side chain of serine or threonine, R₃ is the side chain of glutamine, R₄ is the side chain of isoleucine or D-allo isoleucine and/or R₅ is the side chain of isoleucine or D-allo isoleucine. In additional antibiotics of formula II or II′, R₆ is H or a C₁ to C₉ linear or branched alkyl group, preferably, methyl.

In further embodiments, the antibiotics of the invention have Formula III or III′:

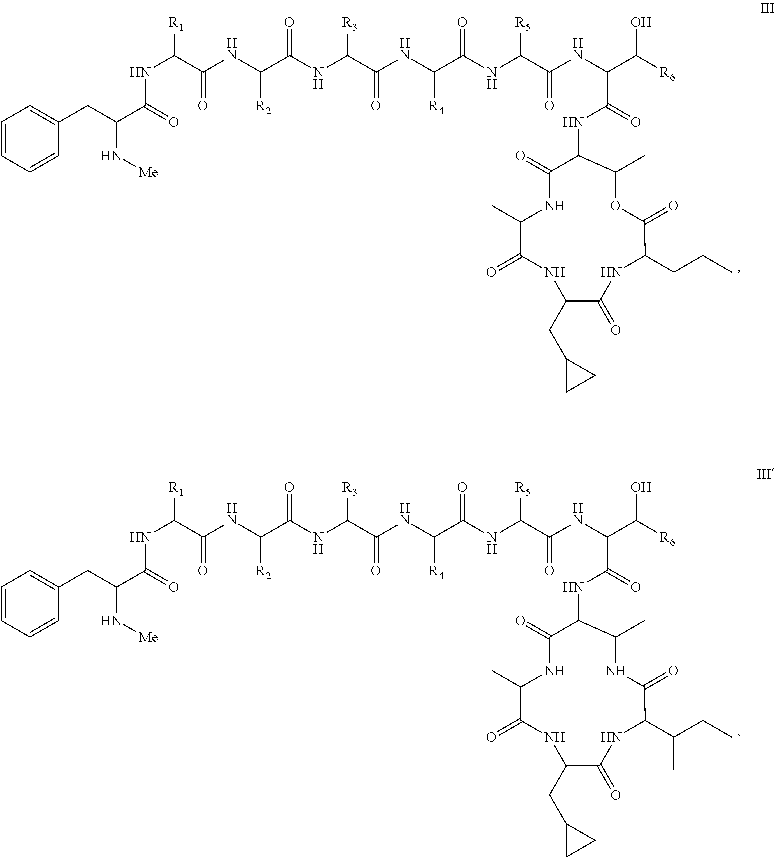

In some antibiotics of Formula III or III′, each of R₁ to R₅ is a side chain of amino acids, including natural or unnatural amino acids. In preferred embodiments of Formula III or III′, each of R₁ to R₅ is independently a side chain of leucine, isoleucine, glutamine, serine, threonine, D-allo isoleucine, lysine, or arginine. In specific embodiments of the antibiotics of Formula III or III′: R₁ is the side chain of isoleucine, R₂ is the side chain of serine or threonine, R₃ is the side chain of glutamine, R₄ is the side chain of isoleucine or D-allo isoleucine and/or R₅ is the side chain of isoleucine or D-allo isoleucine. In additional antibiotics of formula III or III′, R₆ is H or a C₁ to C₉ linear or branched alkyl group, preferably, methyl.

In further embodiments, the antibiotics of the invention have Formula IV or IV′:

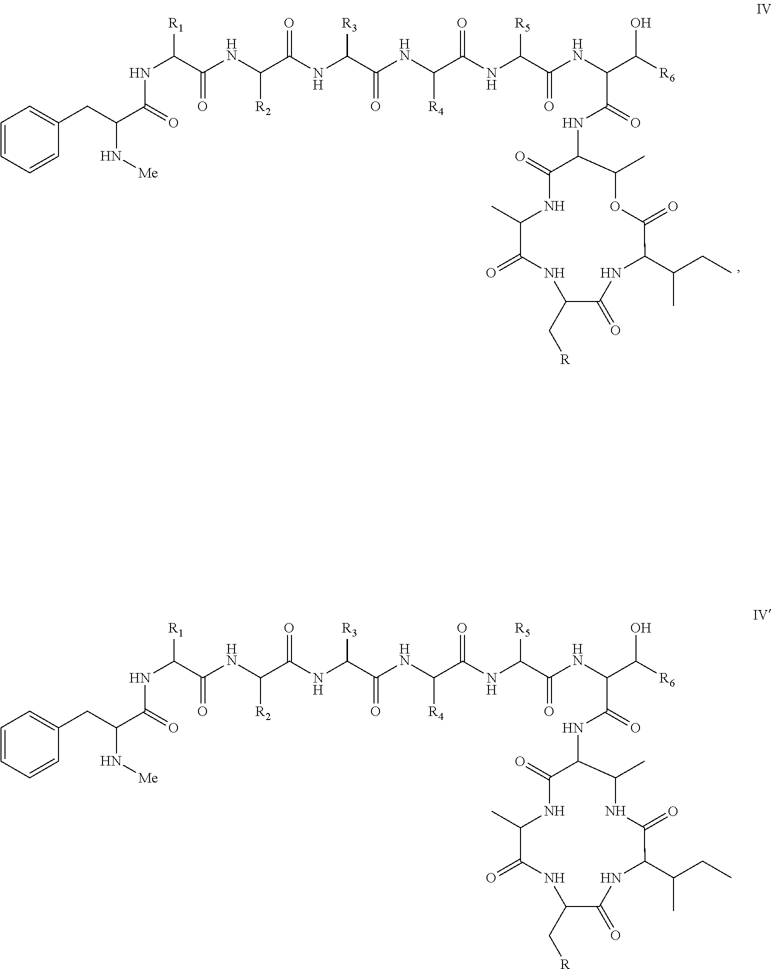

In some antibiotics of Formula IV or IV′, each of R₁ to R₅ is a side chain of amino acids, including natural or unnatural amino acids. In preferred embodiments of Formula IV or IV′, each of R₁ to R₅ is independently a side chain of leucine, isoleucine, glutamine, serine, threonine, D-allo isoleucine, lysine, or arginine. In specific embodiments of the antibiotics of Formula IV or IV′: R₁ is the side chain of isoleucine, R₂ is the side chain of serine or threonine, R₃ is the side chain of glutamine, R₄ is the side chain of isoleucine or D-allo isoleucine and/or R₅ is the side chain of isoleucine or D-allo isoleucine. In additional antibiotics of formula IV or IV′, R₆ is H or a C₁ to C₉ linear or branched alkyl group, preferably, methyl. In further embodiments, R is H or C₂-C₈ linear or branched alkyl chains.

In certain embodiments, the antibiotics of the invention have Formula V or V′:

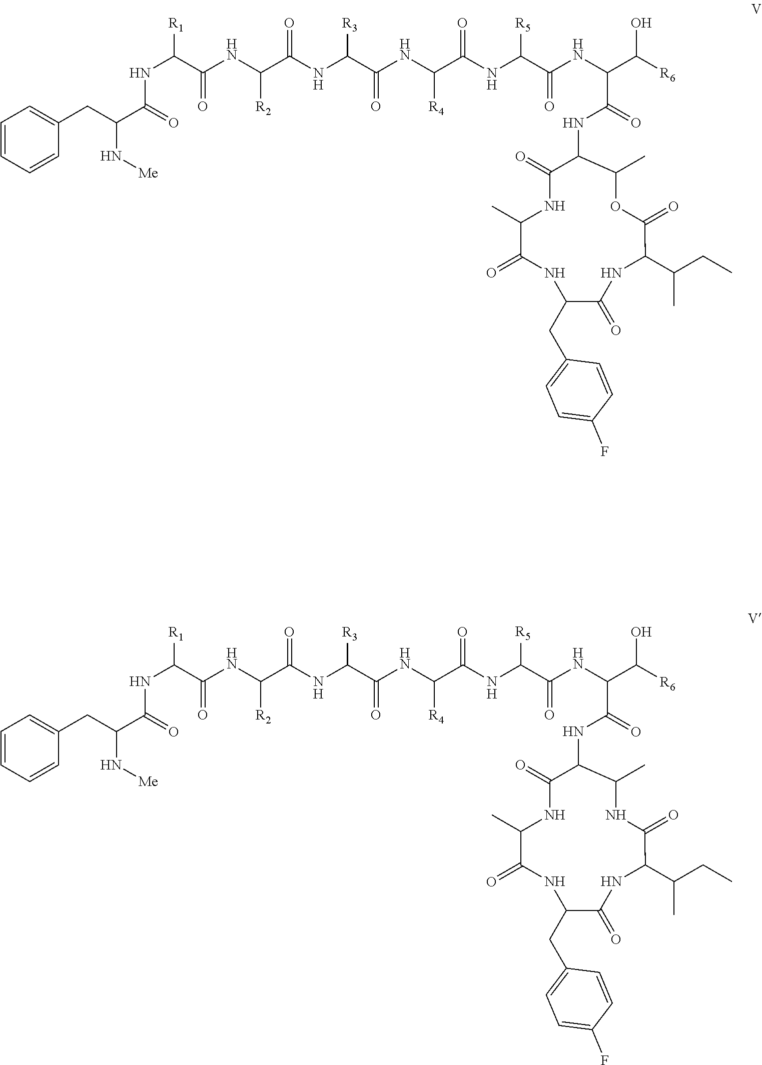

In some antibiotics of Formula V or V′, each of R₁ to R₅ is a side chain of amino acids, including natural or unnatural amino acids. In preferred embodiments of Formula V or V′, each of R₁ to R₅ is independently a side chain of leucine, isoleucine, glutamine, serine, threonine, D-allo isoleucine, lysine, or arginine. In specific embodiments of the antibiotics of Formula V or V′: R₁ is the side chain of isoleucine, R₂ is the side chain of serine or threonine, R₃ is the side chain of glutamine, R₄ is the side chain of isoleucine or D-allo isoleucine and/or R₅ is the side chain of isoleucine or D-allo isoleucine. In additional antibiotics of formula V or V′, R₆ is H or a C₁ to C₉ linear or branched alkyl group, preferably, methyl.

In even further embodiments, the antibiotics of the invention have Formula VI or VI′:

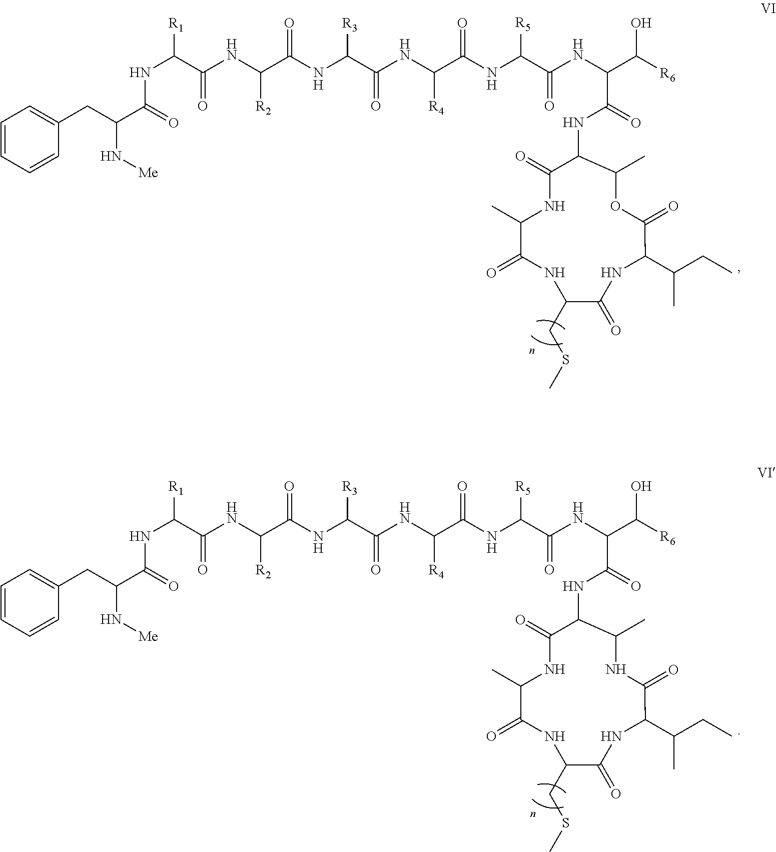

In some antibiotics of Formula VI or VI′, each of R₁ to R₅ is a side chain of amino acids, including natural or unnatural amino acids. In preferred embodiments of Formula VI or VI′, each of R₁ to R₅ is independently a side chain of leucine, isoleucine, glutamine, serine, threonine, D-allo isoleucine, lysine, or arginine. In specific embodiments of the antibiotics of Formula VI or VI′: R₁ is the side chain of isoleucine, R₂ is the side chain of serine or threonine, R₃ is the side chain of glutamine, R₄ is the side chain of isoleucine or D-allo isoleucine and/or R₅ is the side chain of isoleucine or D-allo isoleucine. In additional antibiotics of formula VI or VI′, R₆ is H or a C₁ to C₉ linear or branched alkyl group, preferably, methyl. In specific embodiments of the antibiotics of Formula VI or VI′: n=1-3.

The antibiotics of the invention can be prepared using solution phase synthesis/solid phase synthesis hybrid method. In such hybrid method, L-allo-End in the natural teixobactin (FIG. 1) is replaced by other natural or unnatural amino acids. Also, the residues in the linear peptide portion of teixobactin can be exchanged with other amino acids.

Based on various antibiotics of formulas I to VI′ and different substituents at R₁ to R₆ and R positions, the invention provides a range of antibiotics. Exemplary antibiotics based on various substituents were synthesized and characterized.

For example, end of teixobactin core of FIG. 1 was replaced with other unnatural hydrophobic amino acids and the resulting antibiotic exhibited antibacterial activity against methicillin-susceptible S. aureus strain ATCC29213 (MSSA), and a methicillin-resistant S. aureus (MRSA) clinical isolate (MICS: 0.25-1 μg/mL). The antibacterial activity of this antibiotic against such MSSA and MRSA strains are comparable or superior to teixobactin.

Antibacterial activities of additional such examples are provided in Table 1 below:

TABLE 1
Minimum inhibitory concentration (MIC)(μg/mL) of various
examples of the antibiotics of the invention. The amino acid
substitutions in the antibiotics provided in this Table 1 are
indicated by substituted amino acid compared to the teixobactin
of FIG. 1 and the position of the substituted amino acid. For
example, Thr3-teixobactin indicates that third amino acid in
the teixobactin of FIG. 1 is substituted by threonine.

| Compounds | MRSA(SA11I4) | SA (ATCC29213) |
|---|---|---|
| Met10-teixobactin/ | 1 | 0.25 |
| Methibactin | | |
| Phe(4-F)10- | 1 | 0.5 |
| teixobactin/ | | |
| Flurobactin | | |
| Nle10-teixobactin | 2 | 0.5 |
| Nva10-teixobactin | 1 | 0.25 |
| Chg10-teixobactin | 1 | 0.25 |
| Cha10-teixobactin | 2 | 0.5 |
| β-cyclopropyl-Ala10- | 2 | 0.5 |
| teixobactin | | |
| Teixobactin | 2 | 0.5 |

In Table 1, MICS of the teixobactin analogues are in μg mL⁻¹.

As shown in table 1, four analogues (Met10-teixobactin, Phe(4-F)10-teixobactin, Nva10-teixobactin and Chg10-teixobactin) showed two times higher activities than teixobactin. Another three analogues (Nle10-teixobactin, Cha10-teixobactin and β-cyclopropyl-Ala10-teixobactin) also exhibited comparable activities as teixobactin.

The following compounds (T83-T88) are also prepared and the activities of these compounds are provided in Table 2 below:

TABLE 2
Minimum inhibitory concentration (MIC) (μg/ml) of compounds T83-T88.

| | Strains | | | | | | | | |
|---|---|---|---|---|---|---|---|---|---|
| | | | | | | Streptococcus | | | |
| | MRSA | | | | SA | faecalis | Enterococcus | | |
| | SA11I4 | SA11 | SA86 | SA88 | ATCC29213 | SF | ET6 | ET60 | ATCCET |
| Teixobactin | 2 | 2 | 2 | 1 | 1 | 2 | 2 | 2 | 2 |
| T83 | 1 | 0.5 | 1 | 0.5 | 0.5 | 0.5 | 1 | 0.5 | 1 |
| T84 | 1 | 0.5 | 1 | 1 | 0.5 | 1 | 1 | 1 | 1 |
| T85 | 1 | 1 | 2 | 2 | 0.5 | 1 | 2 | 2 | 2 |
| T86 | 2 | 1 | 2 | 2 | 1 | 1 | 2 | 2 | 2 |
| T87 | 2 | 0.5 | 2 | 1 | 1 | 1 | 1 | 0.5 | 1 |
| T88 | 8 | 4 | 4 | 4 | 4 | 1 | 2 | 1 | 2 |

In Table 2, MICS of the teixobactin analogues are in μg mL⁻¹

As shown in Table 2, all of these analogues exhibited very potent anti-bacterial activities against MRSA, SA, Streptococcus faecalis and Enterococcus strains. Analogues T83, T84, T85 and T86 , in which the ester bond was replaced by an amide bond in the cyclic tetrapeptide in these analogues, showed 2-4 times higher activities than teixobactin. Analogue T87 also showed 2-4 times higher activities than teixobactin. Although the activities of analogue T88 against MRSA and SA strains were about 4 times lower than teixobactin, the activities against Streptococcus faecalis and Enterococcus strains were better than teixobactin.

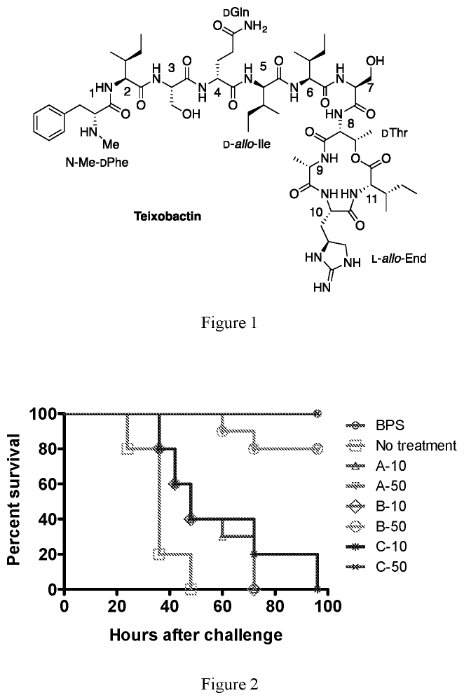

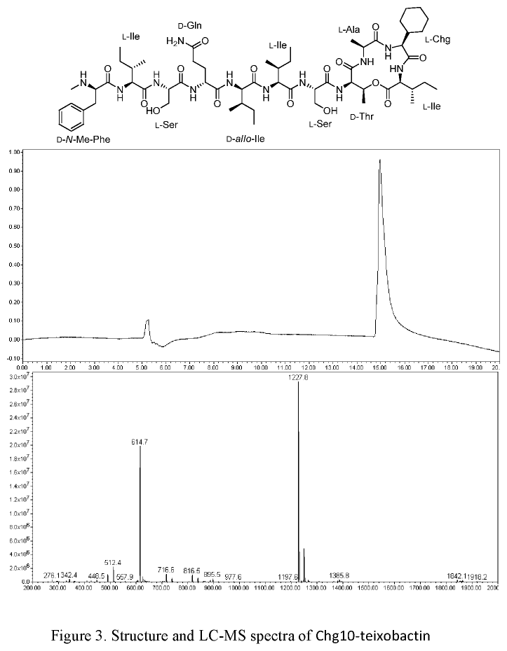

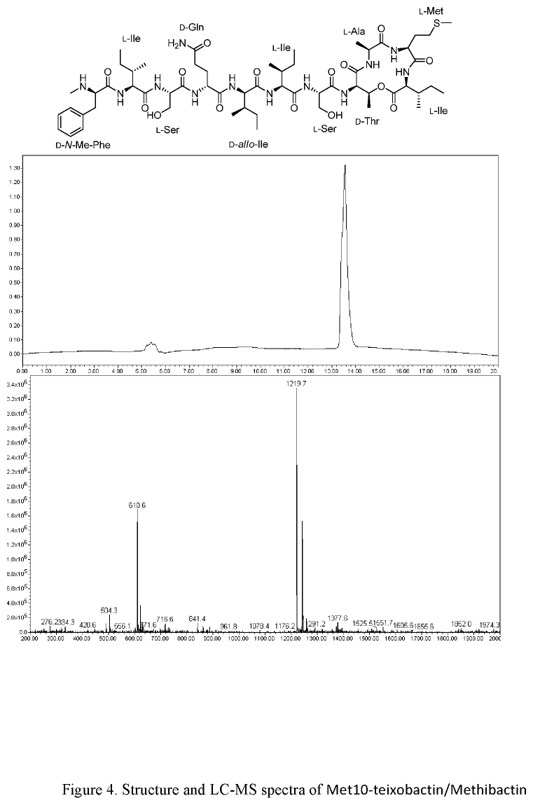

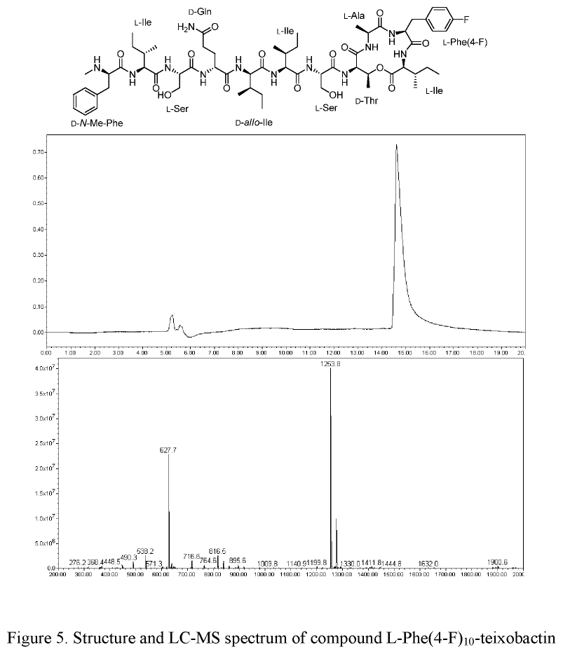

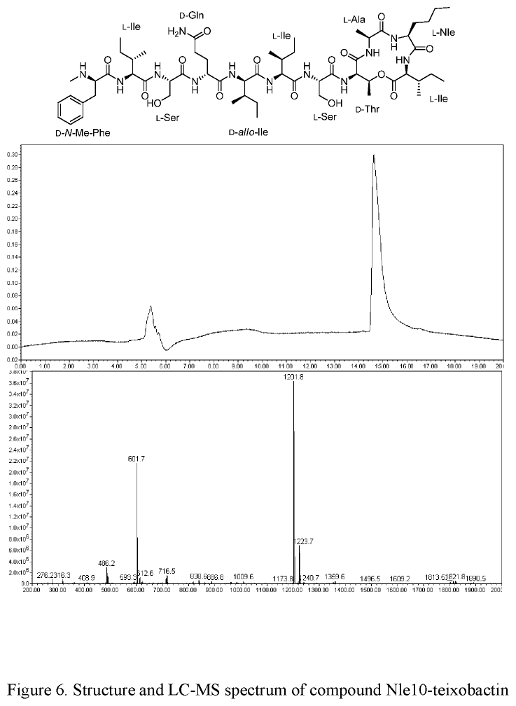

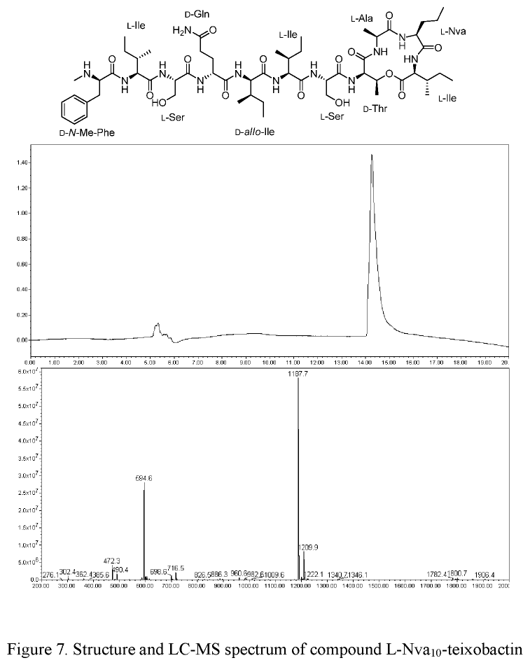

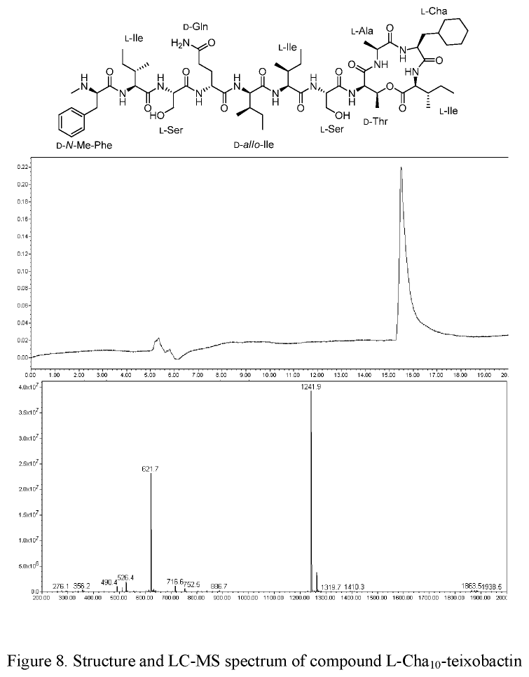

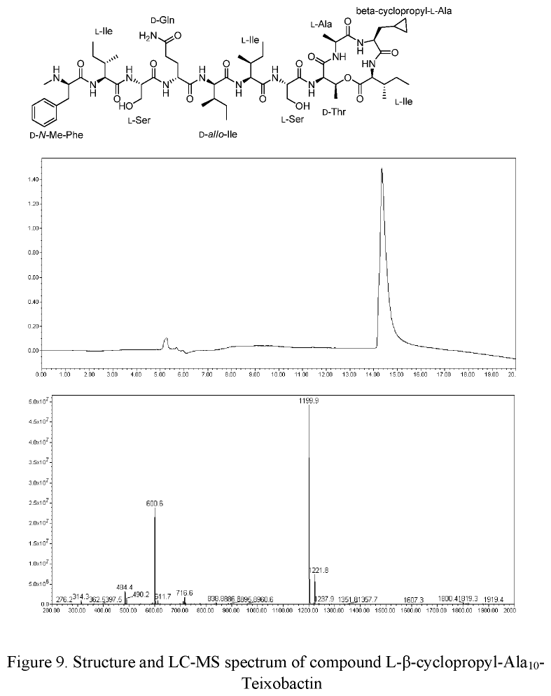

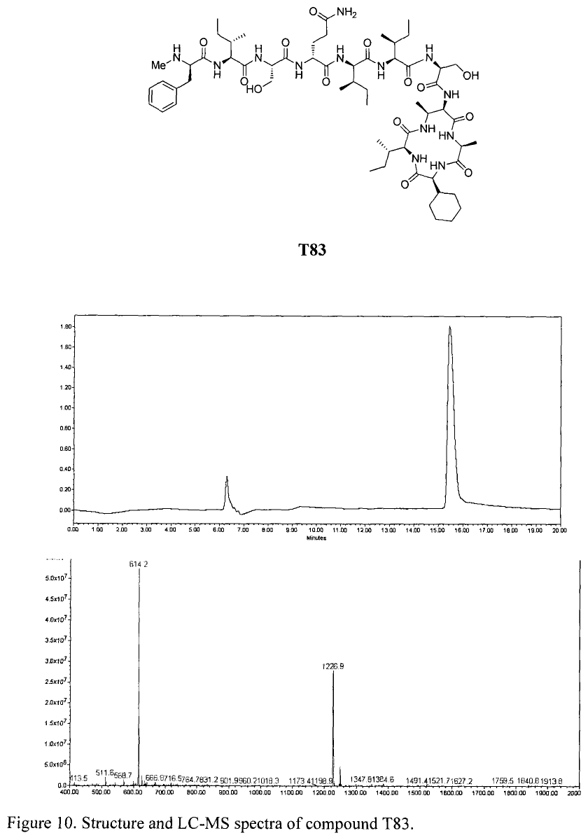

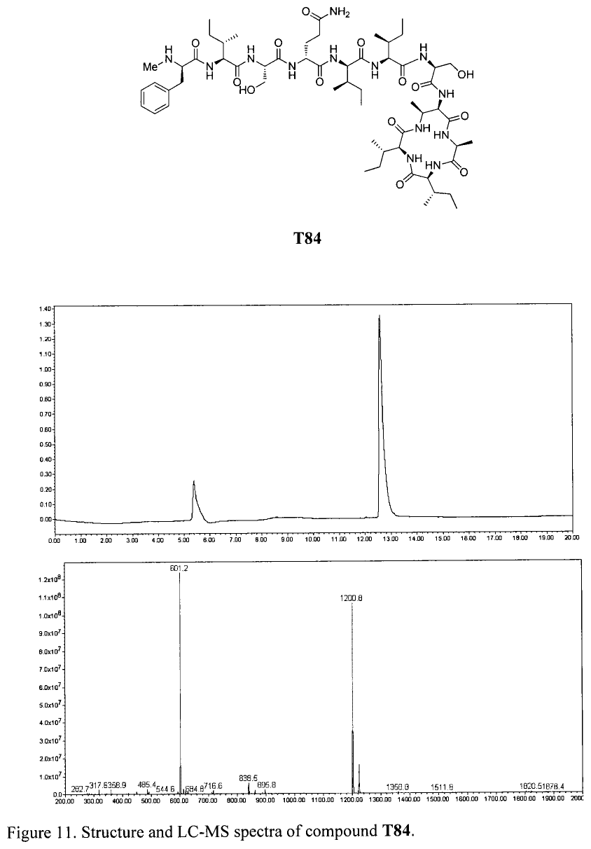

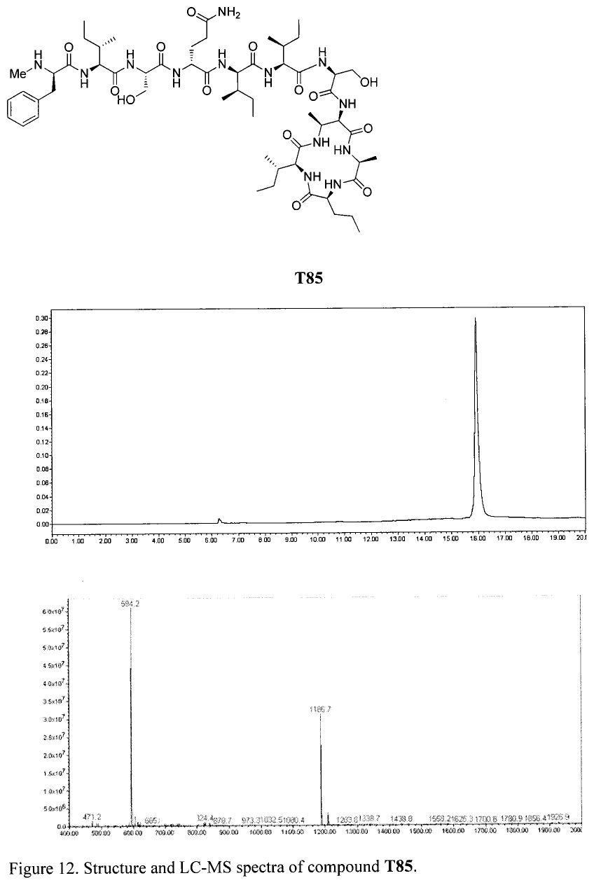

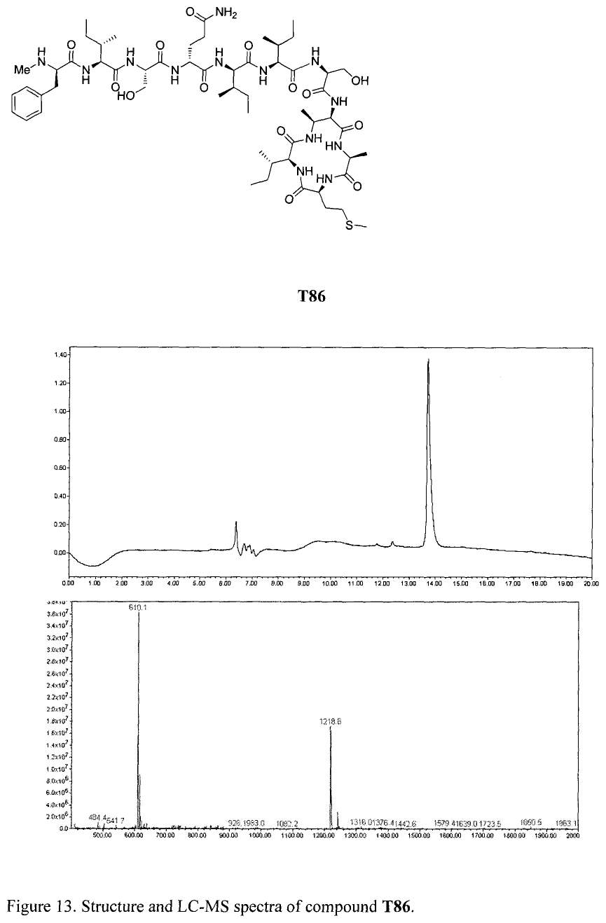

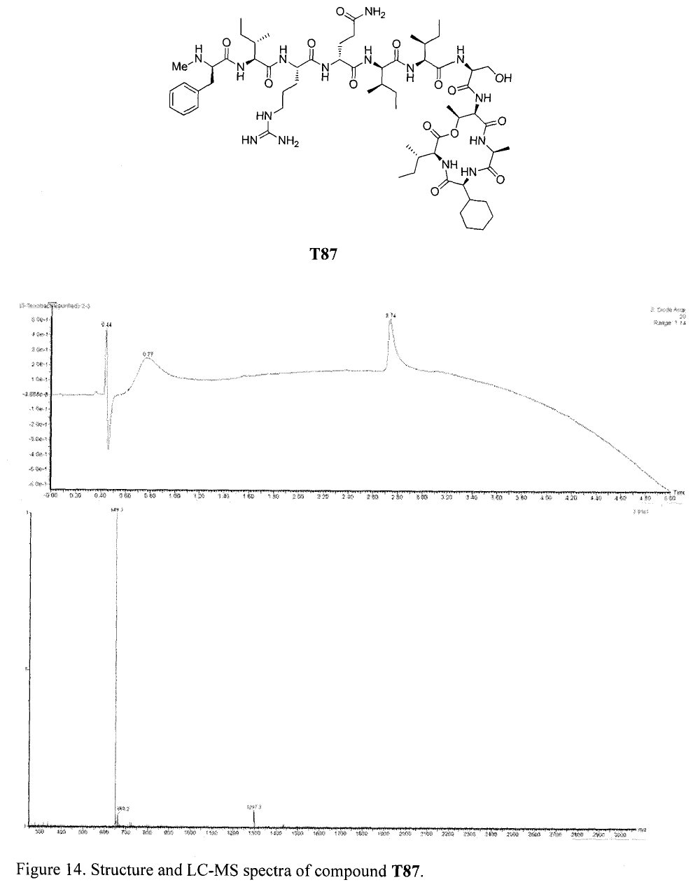

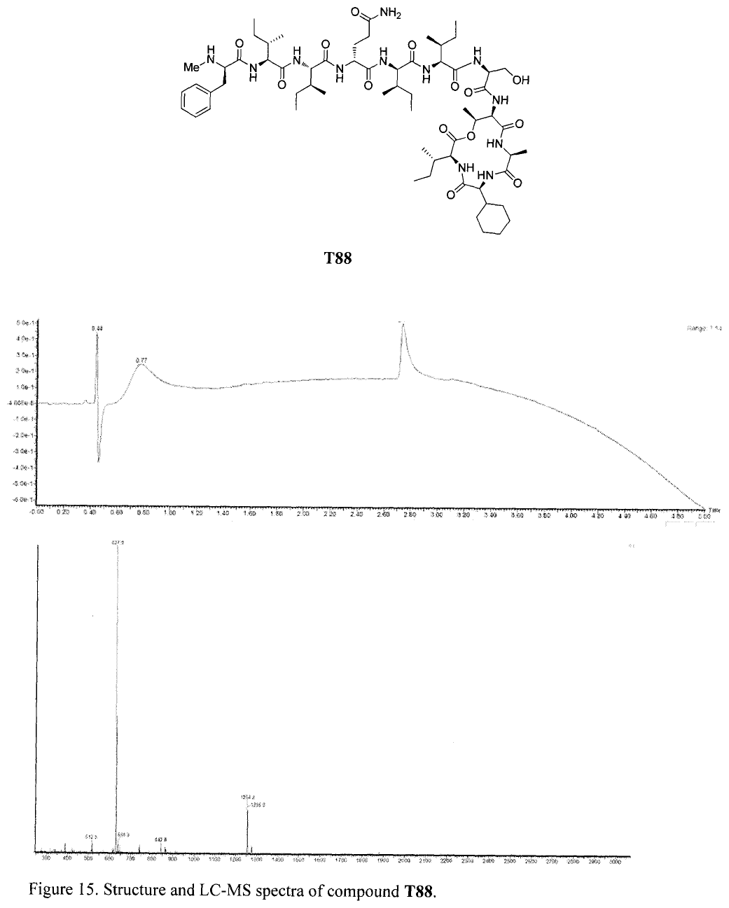

Exemplary in vivo antibacterial activity: Chg10-Teixobactin was studied in wax worm survival models infected with S. aureus strain ATCC43300. Teixobactin were used as controls. As seen, with one dose treatment, Chg10-Teixobactin exhibited improved antibacterial activities. At a dose of 50 mg/kg, Chg10-Teixobactin protected 100% infected wax worms (See FIG. 2).

Some embodiments of the invention provide methods of treating an infection in a subject by administering the antibiotics of the invention to the subject. In some embodiments, the subject is a plant or an animal, preferably, a mammal, and more preferably, a human. In certain embodiments, the infection is caused by an agent such as, but not limited to, a bacterium, a fungus, a virus, a protozoan, a helminth, a parasite, and combinations thereof.

Accordingly, certain embodiments of the methods of treating an infection comprise administering to a subject a therapeutically effective amount of antibiotics described herein, for example, an antibiotic Formula I or I′, II or II′, III or III′ or IV or IV′, thereby treating the infection in the subject.

In particular embodiments, the agent is a bacterium, such as a Gram-positive bacterium or a Gram-negative bacterium. Non-limiting examples of Gram-positive bacteria include Streptococcus, Staphylococcus, Enterococcus, Corynebacteria, Listeria, Bacillus, Erysipelothrix, and Actinomycetes. Non-limiting examples of Gram-negative bacteria include Helicobacter, Neisseria, Campylobacter, Enterobacter, Pseudomonas, Klebsiella, Pasteurella, Bacteroides, Streptobacillus, Leptospira, Salmonella, and Citrobacter.

In some embodiments, the antibiotics of Formula I or I′, II or II′, III or III′ or IV or IV′ are used to treat an infection by one or more of: Helicobacter pylori, Legionella pneumophilia, Mycobacterium tuberculosis, Mycobacterium avium, Mycobacterium intracellulare, Mycobacterium kansaii, Mycobacterium gordonae, Mycobacteria sporozoites, Staphylococcus aureus, Staphylococcus epidermidis, Neisseria gonorrhoeae, Neisseria meningitidis, Listeria monocytogenes, Streptococcus pyogenes (Group A Streptococcus), Streptococcus agalactiae pyogenes (Group B Streptococcus), Streptococcus dysgalactia, Streptococcus faecalis, Streptococcus bovis, Streptococcus pneumoniae, pathogenic Campylobacter sporozoites, Enterococcus sporozoites, Haemophilus influenzae, Pseudomonas aeruginosa, Bacillus anthracis, Bacillus subtilis, Escherichia coli, Corynebacterium diphtheriae, Corynebacterium jeikeium, Corynebacterium sporozoites, Erysipelothrix rhusiopathiae, Clostridium perfringens, Clostridium tetani, Clostridium difficile, Enterobacter aerogenes, Klebsiella pneumoniae, Pasturella multocida, Bacteroides thetaiotamicron, Bacteroides uniformis, Bacteroides vulgatus, Fusobacterium nucleatum, Streptobacillus moniliformis, Leptospira, and Actinomyces israelli. In particular, bacterium is methicillin resistant S. aureus or B. anthracis.

In other embodiments, the antibiotics described herein can be used to treat viral infections. Non-limiting examples of infectious viruses that may be treated by the antibiotics of Formula I or I′, II or II′, III or III′ or IV or IV′ include: Retroviridae (e.g., human immunodeficiency viruses, such as HIV-1 (also referred to as HTLV-III, LAV or HTLV-III/LAV), or HIV-III; and other isolates, such as HIV-LP; Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses, severe acute respiratory syndrome (SARS) virus); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arenaviridae (hemorrhagic fever viruses); Reoviridae (e.g., reoviruses, orbiviurses and rotaviruses); Birnaviridae; Hepadnaviridae (e.g., Hepatitis B virus); Parvoviridae (parvoviruses); Papovaviridae (papilloma viruses, polyoma viruses); Adenoviridae (most adenoviruses); Herpesviridae (e.g., herpes simplex virus (HSV) 1 and 2, varicella zoster virus, cytomegalovirus (CMV), herpes viruses); Poxviridae (e.g., variola viruses, vaccinia viruses, pox viruses); and Iridoviridae (e.g., African swine fever virus); and unclassified viruses (e.g., the etiological agents of Spongiform encephalopathies, the agent of delta hepatitis (thought to be a defective satellite of hepatitis B virus), the agents of non-A, non-B hepatitis (class 1=internally transmitted; class 2=parentally transmitted, i.e., Hepatitis C); Norwalk and related viruses, and astroviruses). In specific embodiments, the antibiotics of Formula I or I′, II or II′, III or III′ or IV or IV′ are used to treat an influenza virus, human immunodeficiency virus, or herpes simplex virus.

In yet other embodiments, the antibiotics described herein are useful in treating infections caused by protozoans. Non-limiting examples of protozoa that can be inhibited by the antibiotics of Formula I or I′, II or II′, III or III′ or IV or IV′ include, but are not limited to, Trichomonas vaginalis, Giardia lamblia, Entamoeba histolytica, Balantidium coli, Cryptosporidium parvum and Isospora belli, Trypansoma cruzi, Trypanosoma gambiense, Leishmania donovani, and Naegleria fowleri.

In certain embodiments, the antibiotics described herein are useful in treating infections caused by helminths. Non-limiting examples of helminths that can be inhibited by the antibiotics of Formula I or I′, II or II′, III or III′ or IV or IV′ include, but are not limited to: Schistosoma mansoni, Schistosoma cercariae, Schistosoma japonicum, Schistosoma mekongi, Schistosoma hematobium, Ascaris lumbricoides, Strongyloides stercoralis, Echinococcus granulosus, Echinococcus multilocularis, Angiostrongylus cantonensis, Angiostrongylus constaricensis, Fasciolopis buski, Capillaria philippinensis, Paragonimus westermani, Ancylostoma dudodenale, Necator americanus, Trichinella spiralis, Wuchereria bancrofti, Brugia malayi, and Brugia timori, Toxocara canis, Toxocara cati, Toxocara vitulorum, Caenorhabiditis elegans, and Anisakis spp.

In some embodiments, the antibiotics described herein are useful in treating disorders caused by parasites. Non-limiting examples of parasites that can be inhibited by the antibiotics of Formula I or I′, II or II′, III or III′ or IV or IV′ include, but are not limited to, Plasmodium falciparum, Plasmodium yoelli, Hymenolepis nana, Clonorchis sinensis, Loa boa, Paragonimus westermani, Fasciola hepatica, and Toxoplasma gondii. In specific embodiments, the parasite is a malarial parasite.

In further embodiments, the antibiotics of Formula I or I′, II or II′, III or III′ or IV or IV′ may be useful to treat disorders caused by fungi. Non-limiting examples of fungi that may be inhibited by the antibiotics of Formula I or I′, II or II′, III or III′ or IV or IV′ include, but are not limited to, Cryptococcus neoformans, Histoplasma capsulatum, Coccidioides immitis, Blastomyces dermatitidis, Chlamydia trachomatis, Candida albicans, Candida tropicalis, Candida glabrata, Candida krusei, Candida parapsilosis, Candida dubliniensis, Candida lusitaniae, Epidermophyton floccosum, Microsporum audouinii, Microsporum canis, Microsporum canis var. distortum Microsporum cookei, Microsporum equinum, Microsporum ferrugineum, Microsporum falvum, Microsporum gallinae, Microsporum gypseum, Microsporum nanum, Microsporum persicolor, Trichophyton ajelloi, Trichophyton concentricum, Trichophyton equinum, Trichophyton flavescens, Trichophyton gloriae, Trichophyton megnini, Trichophyton mentagrophytes var. erinacei, Trichophyton mentagrophytes var. interdigitale, Trichophyton phaseoliforme, Trichophyton rubrum, Trichophyton rubrum downy strain, Trichophyton rubrum granualr strain, Trichophyton schoenleinii, Trichophyton simii, Trichophyton soudanense, Trichophyton terrestre, Trichophyton tonsurans, Trichophyton vanbreuseghemii, Trichophyton verrucosum, Trichophyton violaceum, Trichophyton yaoundei, Aspergillus fumigatus, Aspergillus flavus, and Aspergillus clavatus.

In yet another embodiment, the present invention relates to a method of inhibiting the growth of an infectious agent, the method comprising contacting the agent with a compound described herein, e.g., a compound of Formula I or I′, II or II′, III or III′ or IV or IV′, thereby inhibiting the growth of the infectious agent.

### Routes of Administration and Dosage Forms

Certain embodiments of the invention provide pharmaceutical compositions comprising antibiotics of the invention. The pharmaceutical compositions of the invention comprise antibiotics of the invention and pharmaceutically acceptable carriers or excipients.

In certain embodiments, the antibiotics may be administered intramuscularly, subcutaneously, intrathecally, intravenously or intraperitoneally by infusion or injection. Solutions of the antibiotics can be prepared in water, optionally mixed with a nontoxic surfactant. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the antibiotics that are adapted for the extemporaneous preparation of sterile injectable or infusible solutions or dispersions, optionally encapsulated in liposomes. Preferably, the ultimate dosage form should be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained by, for example, the formation of liposomes, by the maintenance of the required particle size in the case of dispersions, or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by the use in the compositions of agents delaying absorption, for example, aluminum monostearate and gelatin.

Sterile injectable solutions are prepared by incorporating the antibiotics in the required amount in the appropriate solvent as described herein with various of the other ingredients enumerated herein, as required, preferably followed by filter sterilization. In the case of sterile powders for the preparation of sterile injectable solutions, the preferred methods of preparation are vacuum drying and freeze drying techniques, which yield a powder of the active ingredient plus any additional desired ingredient present in the previously sterile-filtered solutions.

The compositions of the subject invention may also be administered orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet.

For oral therapeutic administration, the antibiotics may be combined with one or more excipients and used in the form of ingestible tablets, buccal tablets, troches, capsules, elixirs, suspensions, syrups, wafers, and the like. Such compositions and preparations should contain at least 0.1% of an antibiotic of the present invention. The percentage of the antibiotics of the invention present in such compositions and preparations may, of course, be varied and may conveniently be between about 2% to about 60% of the weight of a given unit dosage form. The amount of the antibiotics in such therapeutically useful compositions is such that an effective dosage level will be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid, and the like; a lubricant such as magnesium stearate; and a sweetening agent such as sucrose, fructose, lactose, or aspartame, or a flavoring agent such as peppermint, oil of wintergreen, or cherry flavoring may be added.

When the unit dosage form is a capsule, it may contain, in addition to materials of the above type, a liquid carrier, such as a vegetable oil or a polyethylene glycol.

Various other materials may be present as coatings or for otherwise modifying the physical form of the solid unit dosage form. For instance, tablets, pills, or capsules may be coated with gelatin, wax, shellac, or sugar, and the like. A syrup or elixir may contain the active antibiotic, sucrose or fructose as a sweetening agent, methyl and propylparabens as preservatives, a dye, and flavoring such as cherry or orange flavor.

Of course, any material used in preparing any unit dosage form should be pharmaceutically acceptable and substantially non-toxic in the amounts employed.

In addition, the antibiotics may be incorporated into sustained-release preparations and devices. For example, the antibiotics may be incorporated into time release capsules, time release tablets, time release pills, and time release antibiotics or nanoparticles.

Pharmaceutical compositions for topical administration of the antibiotics to the epidermis (mucosal or cutaneous surfaces) can be formulated as ointments, creams, lotions, gels, or as a transdermal patch. Such transdermal patches can contain penetration enhancers such as linalool, carvacrol, thymol, citral, menthol, t-anethole, and the like. Ointments and creams can, for example, include an aqueous or oily base with the addition of suitable thickening agents, gelling agents, colorants, and the like. Lotions and creams can include an aqueous or oily base and typically also contain one or more emulsifying agents, stabilizing agents, dispersing agents, suspending agents, thickening agents, coloring agents, and the like. Gels preferably include an aqueous carrier base and include a gelling agent such as cross-linked polyacrylic acid antibiotic, a derivatized polysaccharide (e.g., carboxymethyl cellulose), and the like.

Pharmaceutical compositions suitable for topical administration in the mouth (e.g., buccal or sublingual administration) include lozenges comprising the composition in a flavored base, such as sucrose, acacia, or tragacanth; pastilles comprising the composition in an inert base such as gelatin and glycerin or sucrose and acacia; and mouthwashes comprising the active ingredient in a suitable liquid carrier. The pharmaceutical compositions for topical administration in the mouth can include penetration enhancing agents, if desired.

Useful solid carriers include finely divided solids such as talc, clay, microcrystalline cellulose, silica, alumina, and the like. Other solid carriers include nontoxic polymeric nanoparticles or microparticles. Useful liquid carriers include water, alcohols, or glycols, or water/alcohol/glycol blends, in which the antibiotics can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of useful dermatological compositions which can be used to deliver the antibiotics to the skin are known in the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508), all of which are hereby incorporated by reference.

The concentration of the therapeutic antibiotics of the invention in such formulations can vary widely depending on the nature of the formulation and intended route of administration. For example, the concentration of the antibiotics in a liquid composition, such as a lotion, can preferably be from about 0.1-25% by weight, or, more preferably, from about 0.5-10% by weight. The concentration in a semi-solid or solid composition such as a gel or a powder can preferably be about 0.1-5% by weight, or, more preferably, about 0.5-2.5% by weight.

Pharmaceutical compositions for spinal administration or injection into amniotic fluid can be provided in unit dose form in ampoules, pre-filled syringes, small volume infusion, or in multi-dose containers, and can include an added preservative. The compositions for parenteral administration can be suspensions, solutions, or emulsions, and can contain excipients such as suspending agents, stabilizing agents, and dispersing agents.

A pharmaceutical composition suitable for rectal administration comprises antibiotics of the present invention in combination with a solid or semisolid (e.g., cream or paste) carrier or vehicle. For example, such rectal compositions can be provided as unit dose suppositories. Suitable carriers or vehicles include cocoa butter and other materials commonly used in the art.

According to one embodiment, pharmaceutical compositions of the present invention suitable for vaginal administration are provided as pessaries, tampons, creams, gels, pastes, foams, or sprays containing antibiotics of the invention in combination with carriers as are known in the art. Alternatively, compositions suitable for vaginal administration can be delivered in a liquid or solid dosage form.

Pharmaceutical compositions suitable for intra-nasal administration are also encompassed by the present invention. Such intra-nasal compositions comprise antibiotics of the invention in a vehicle and suitable administration device to deliver a liquid spray, dispersible powder, or drops. Drops may be formulated with an aqueous or non-aqueous base also comprising one or more dispersing agents, solubilizing agents, or suspending agents. Liquid sprays are conveniently delivered from a pressurized pack, an insufflator, a nebulizer, or other convenient means of delivering an aerosol comprising the antibiotics. Pressurized packs comprise a suitable propellant such as dichlorodifluoromethane, trichlorofluoromethane, dichlorotetrafluoroethane, carbon dioxide, or other suitable gas as is well known in the art. Aerosol dosages can be controlled by providing a valve to deliver a metered amount of the antibiotic.

The antibiotics may be combined with an inert powdered carrier and inhaled by the subject or insufflated.

Pharmaceutical compositions for administration by inhalation or insufflation can be provided in the form of a dry powder composition, for example, a powder mix of the antibiotics and a suitable powder base such as lactose or starch. Such powder composition can be provided in unit dosage form, for example, in capsules, cartridges, gelatin packs, or blister packs, from which the powder can be administered with the aid of an inhalator or insufflator.

The exact amount (effective dose) of the antibiotic will vary from subject to subject, depending on, for example, the species, age, weight, and general or clinical condition of the subject, the severity or mechanism of any infection being treated, the particular agent or vehicle used, the method and scheduling of administration, and the like. A therapeutically effective dose can be determined empirically, by conventional procedures known to those of skill in the art. See, e.g., The Pharmacological Basis of Therapeutics, Goodman and Gilman, eds., Macmillan Publishing Co., New York. For example, an effective dose can be estimated initially either in cell culture assays or in suitable animal models. The animal model may also be used to determine the appropriate concentration ranges and routes of administration. Such information can then be used to determine useful doses and routes for administration in humans. Methods for the extrapolation of effective dosages in mice and other animals to humans are known to the art; for example, see U.S. Pat. No. 4,938,949, which is hereby incorporated by reference. A therapeutic dose can also be selected by analogy to dosages for comparable therapeutic agents.

The particular mode of administration and the dosage regimen will be selected by the attending clinician, taking into account the particulars of the case (e.g., the subject, the disease, the disease state involved, and whether the treatment is prophylactic). Treatment may involve daily or multi-daily doses of compound(s) over a period of a few days to months, or even years.

In general, however, a suitable dose will be in the range of from about 0.001 to about 100 mg/kg of body weight per day, preferably from about 0.01 to about 100 mg/kg of body weight per day, more preferably, from about 0.1 to about 50 mg/kg of body weight per day, or even more preferred, in a range of from about 1 to about 10 mg/kg of body weight per day. For example, a suitable dose may be about 1 mg/kg, 10 mg/kg, or 50 mg/kg of body weight per day.

The antibiotics can be conveniently administered in unit dosage form, containing for example, about 0.05 to about 10000 mg, about 0.5 to about 10000 mg, about 5 to about 1000 mg, or about 50 to about 500 mg of active ingredient per unit dosage form.

The antibiotics can be administered to achieve peak plasma concentrations of, for example, from about 0.25 to about 200 μM, about 0.5 to about 75 μM, about 1 to about 50 μM, about 2 to about 30 μM, or about 5 to about 25 μM. Exemplary desirable plasma concentrations include at least 0.25, 0.5, 1, 5, 10, 25, 50, 75, 100 or 200 μM. For example, plasma levels may be from about 1 to about 100 micromolar or from about 10 to about 25 micromolar. This may be achieved, for example, by the intravenous injection of a 0.05 to 5% solution of the antibiotics, optionally in saline, or orally administered as a bolus containing about 1 to about 100 mg of the antibiotics. Desirable blood levels may be maintained by continuous or intermittent infusion.

The antibiotics can be included in the compositions within a therapeutically useful and effective concentration range, as determined by routine methods that are well known in the medical and pharmaceutical arts. For example, a typical composition can include one or more of the antibiotics at a concentration in the range of at least about 1 mg/ml, preferably at least about 4 mg/ml, more preferably at least 5 mg/ml and most preferably at least 6 mg/ml.

The antibiotics may conveniently be presented in a single dose or as divided doses administered at appropriate intervals, for example, as one dose per day or as two, three, four or more sub-doses per day. The sub-dose itself may be further divided, e.g., into a number of discrete loosely spaced administrations; such as multiple inhalations from an insufflator.

Optionally, the pharmaceutical compositions of the present invention can include one or more other therapeutic agents, e.g., as a combination therapy. The additional therapeutic agent(s) will be included in the compositions within a therapeutically useful and effective concentration range, as determined by routine methods that are well known in the medical and pharmaceutical arts. The concentration of any particular additional therapeutic agent may be in the same range as is typical for use of that agent as a monotherapy, or the concentration may be lower than a typical monotherapy concentration if there is a synergy when combined with antibiotics of the present invention.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Synthesis Process for the Compounds of the Invention

### Chemistry

Fmoc-Ile-DThr(NH-Alloc)-OH, Boc-D-N-methyl-Phe-OH and Boc-Ile-SAL^{off} were prepared as reported^{1,2}. All the commercial amino acids and coupling reagents were used without further purification. All HPLC grade (DUKSAN) and analytical grade (RCI) solvents were used as received unless otherwise noted. Anhydrous dichloromethane (DCM) was distilled in the presence of calcium hydride (CaH₂). Analytical reverse-phase HPLC was performed on a waters system equipped with a Vydac 218™ C18 column (5 μm, 4.6×250 mm) using specified linear gradients of acetonitrile (0.1% TFA) in water (0.1% TFA) with a flow rate of 0.6 mL/min. Preparative reverse-phase HPLC was performed on a Waters system equipped with a Vydac 218TP™ C18 column (10 nm, 10×250 mm) using specified linear gradients of acetonitrile (0.1% TFA) in water (0.1% TFA) with a flow rate of 10.0 mL/min.

### General Fmoc-SPPS (Solid Phase Peptide Synthesis) Procedure.

100 mg 2-chlorotrityl resin (0.5 mmol/g) was swollen in dry DCM for 30 min and treated with the first building block (2.0 equiv) and DIEA (4.0 equiv) in dry DCM. After it was shook for 1 hour, 80 μL MeOH was added to cap the unreacted resin for another 20 min. The loaded resin was washed by DCM (3×2 mL) and DMF (3×2 mL). Fmoc deprotection was achieved by shaken with 2mL 20% solution of piperidine in DMF for 20 min. The following Fmoc- or Boc-amino acids (4.0 equiv) was coupled using HATU (4.0 equiv) as coupling reagent and DIEA (8.0 equiv) as base. The mixture was shaken in DMF for 1 hour. After each Fmoc deprotection and coupling reaction, the resin was washed by DMF (3×2 mL), DCM (3×2 mL) and DMF (3×2 mL).

### Alloc Deprotection.

The loaded resin was washed by DCM (3×2 mL) and then a solution of Pd(PPh₃)₄ (1.0 equiv) and phenylsilane (25 equiv) in 2 mL anhydrous DCM was added. The mixture was shaken for 1 hour under the protection of dry argon. After Alloc deprotection was completed, the resin was washed by DMF (3×2 mL), DCM (3×2 mL) and DMF (3×2 mL).

### Peptide Cleavage.

After coupling of the last building block, the resin was washed by DCM (3×2 mL), DMF (3×2 mL) and DCM (5×2 mL). Then a cocktail of DCM/AcOH/TFE((v/v/v=8:1:1) was added to the resin and shaken for 1.5 hours. Then the resin was filtrated off and rinsed with DCM (5×2 mL). The combined filtrates were concentrated under low pressure and azeotroped several times with DCM to remove the Acetic acid. The side-chain-protected peptides were obtained as white solid.

### Cyclization and Side Chain Deprotection.

The side-chain-protected peptides (1.0 equiv) was dissolved in anhydrous DCM at a concentration of 0.1 mmol/L. A solution of HOAT (6.0 equiv), Oxyma pure (6.0 equiv) and DIEA (12.0 equiv) in anhydrous DCM was added and stirred at 0° C. for 15 min. Then HATU (10.0 equiv) was added. The resulting reaction mixture was warmed up to room temperature slowly and continued to be stirred for 24 hours. Complete cyclization reactions were confirmed by LC-MS monitoring.

DCM was evaporated in vacuo and the residue was treated with a cocktail of TFA/TIPS/H₂O (v/v/v=95:2.5:2.5) for 1.5 hours. TFA/TIPS/H₂O was blown away by a condensed air stream and the residue was precipitated and washed using cold ethyl ether (20 mL×3) to yield the crude cyclic unprotected-peptide.

The crude cyclic peptide was dissolved in a CH₃CN/H₂O (v/v=1:1) solution and purified by preparative HPLC (5-50% CH₃CN [0.1% TFA] in H₂O [0.1% TFA] over 30 min) to afford pure cyclic peptides.

### Peptide 1-6 SAL Ester Synthesis.

The peptide 1-6 SAL ester was synthesized through a “n+1” strategy. The “n” (side-chain-protected peptide 1-5) was prepared according to the general Fmoc-SPPS and cleavage procedure.

The compound Boc-Ile-SAL^{off} (1.0 equiv) was dissolved in 4.0N HCl solution in dioxane (10.0 equiv) and stirred for 1 hour at room temperature. Then the solvent was blown away by a condensed air stream and the residue was precipitated and washed using cold diethyl ether (20 mL×3) to yield the crude “1”.

The “n” (1.0 equiv) and “1” (3.0 equiv) were dissolved in a mixture of CHCl₃/TFE (v/v=3:1), then EDC (3.0 equiv) and HOOBT (3.0 equiv) were added as coupling reagent. The resulting reaction mixture was stirred at room temperature for 6 hours. After the completion of coupling reaction, CHCl₃/TFE was blown off under a stream of condensed air. The residue was treated by TFA/TIPS/H₂O (v/v/v=95:2.5:2.5) for 1 hour and pyruvic acid (100.0 equiv) for another 2 hours. The resulting mixture was triturated with cold diethyl ether to give a suspension. After centrifugation, the crude peptide was dissolved in a CH₃CN/H₂O (v/v=1:1) solution and purified by preparative HPLC (30-80% CH₃CN [0.1% TFA] in H₂O [0.1% TFA] over 30 min) to afford pure peptide 1-6 SAL ester as a white solid.

### Ser Ligation.

The cyclic peptides (1.0 equiv) and peptide 1-6 SAL eater (1.2 equiv) were dissolved in a mixture of pyridine/AcOH (mol:mol=6:1) at a concentration of 10.0 mmol/L. The reaction mixture was stirred at room temperature for 10 hours. After the pyridine/AcOH was removed by lyophilization, the residue was treated with TFA/TIPS/H₂O (v/v/v=95:2.5:2.5) for 1 hour. Then the crude peptide was blown-dry under a stream of condensed air and purified by preparative HPLC (20-60% CH₃CN [0.1% TFA] in H₂O [0.1% TFA] over 30 min) to afford teixobactin analogues as white solid.

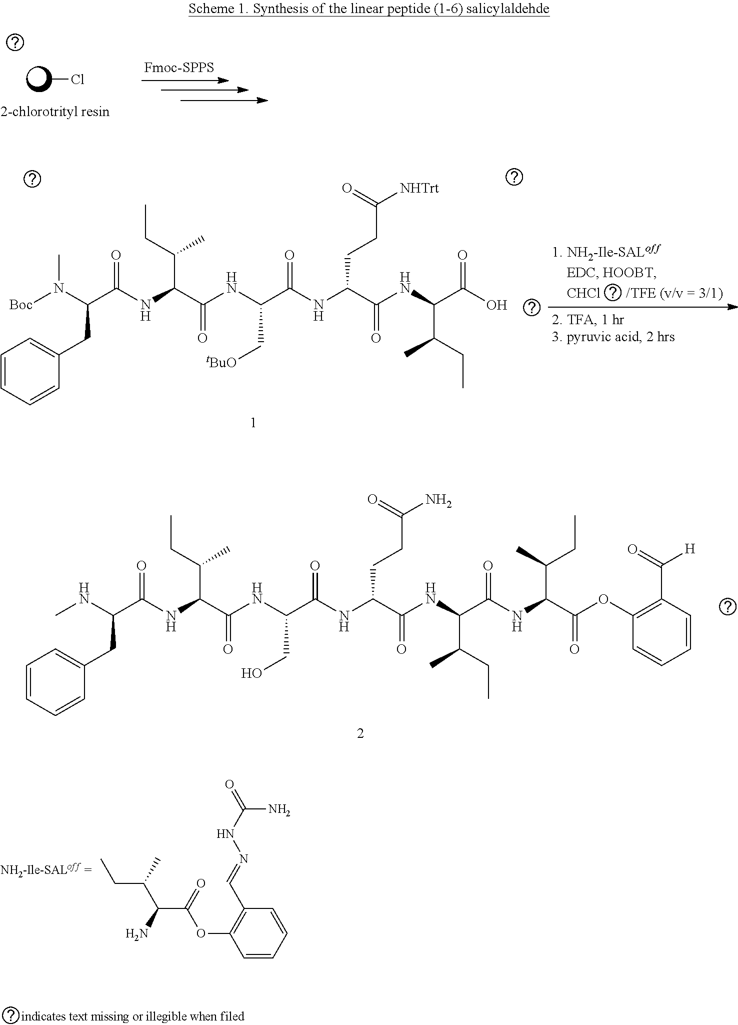

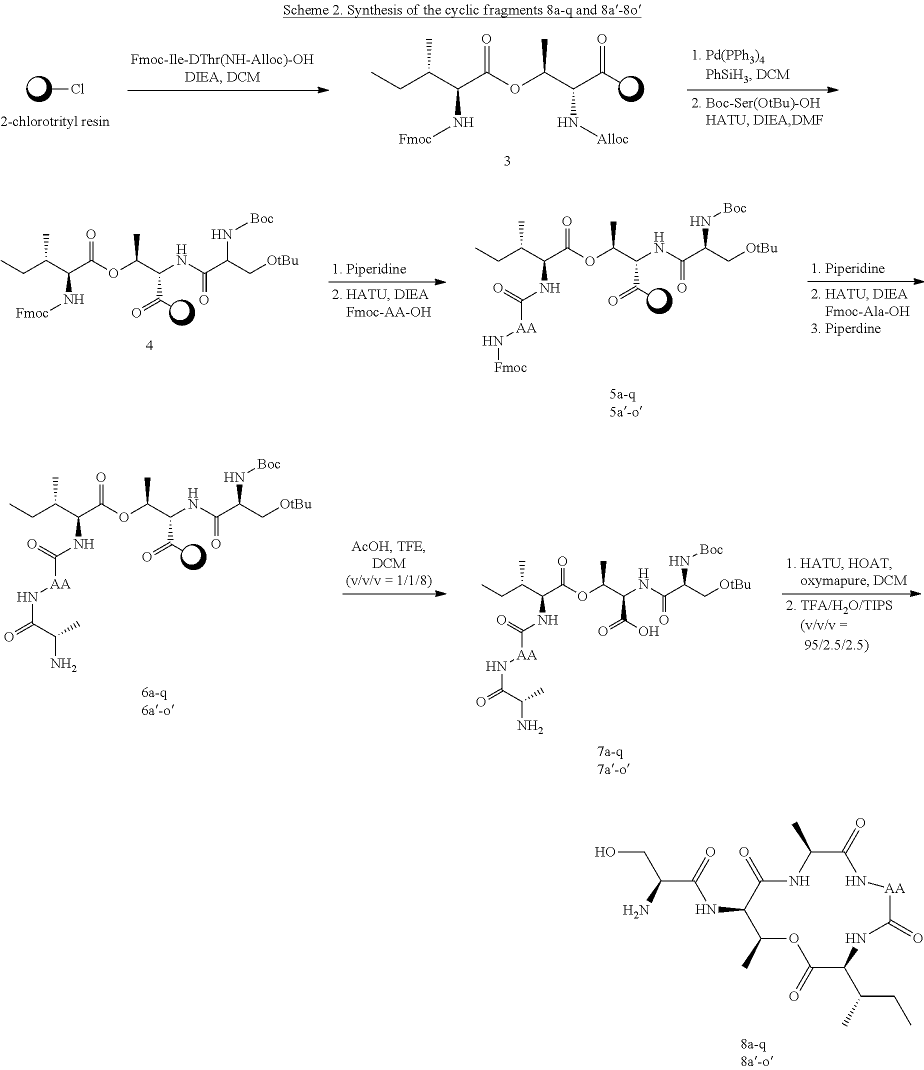

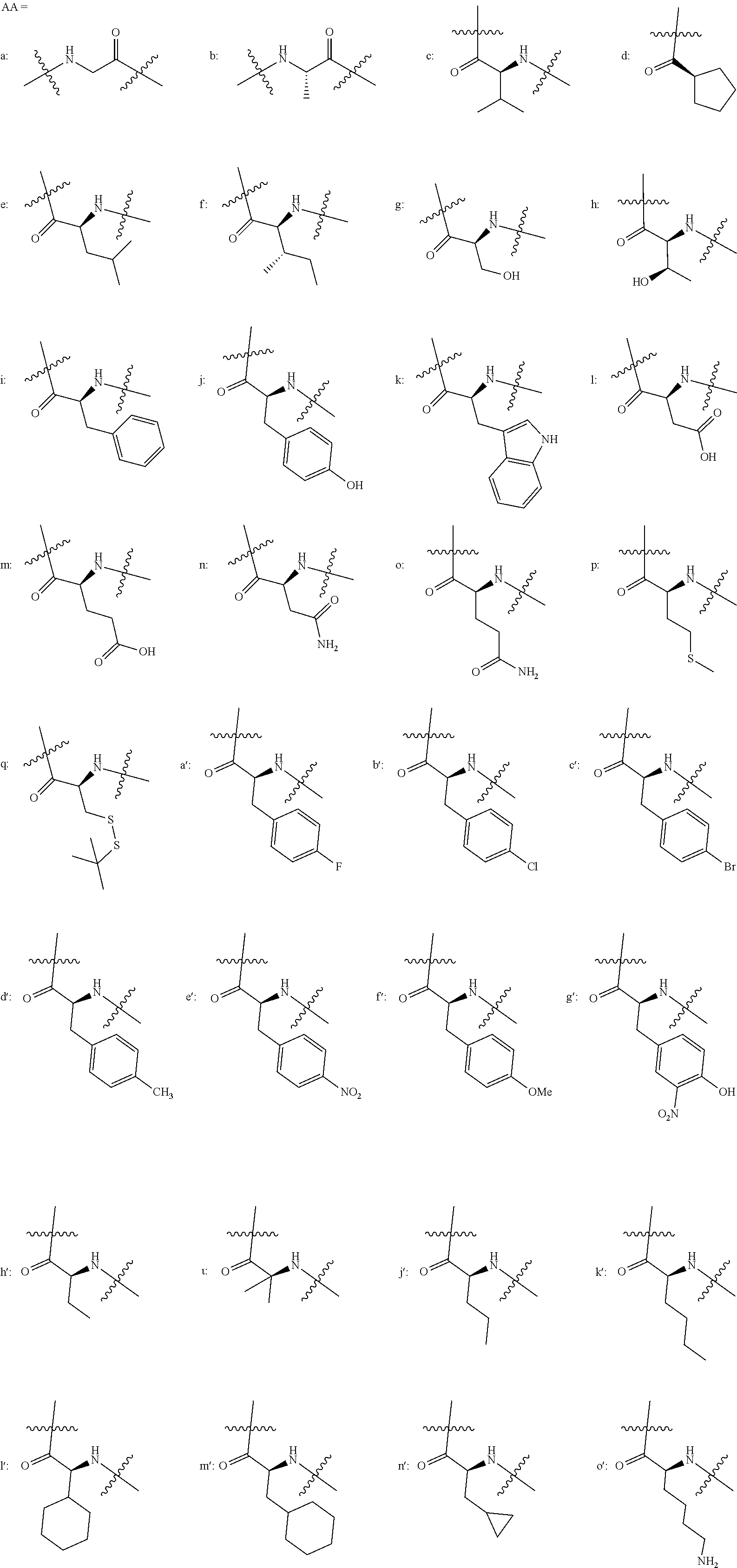

Antibacterial Studies

Susceptibility to teixobactin and its analogues was tested on the selected Gram-positive strains using Tecan Freedom EVO high-throughput automated platform, following the standard broth dilution method as described by the Clinical and Laboratory Standards Institute ³. MICS were determined according to CLSI guideline.

### REFERENCES AND NOTES

1. Ling L. L., Schneider T., Peoples A. J., Spoering A. L., Engels I., Conlon B. P., Muller A., Schaberle T. F., Hughes D. E., Epstein S., Jones M., Lazarides L., Steadman V. A., Cohen D. R., Felix C. R., Fetterman K. A., Millett W. P., Nitti A. G., Zullo A. M., Chen C., Lewis K. Nature 2015; 517: 455-459.

2. Zhang Y., Xu C., Lam H. Y., Lee C. L., Li X. Proc. Natl. Acad. Sci. U. S. A. 2013; 110: 6657-6662.

3. CLSI. CLSI document M 100-S26, Wayne, Pa.: Clinical and Laboratory Standards Institute (2016).

### LIST OF ABBREVIATIONS

AcOH: Acetic acid

### Alloc: Allyloxycabonyl

Boc: tert-Butyloxycarbonyl

### DCM: Dichloromethane

### DIEA: N,N-Diisopropylethylamine

### DMF: NN-Dimethylformamide

EDC: 1-Ethyl-3-(3-dimethylaminopropyl)carbodiimide

### Fmoc: 9-Fluorenylmethoxycarbonyl

HATU: O-(7-Azabenzotriazol-1-yl)-N,N,N ‘,N ’-tetramethyluronium
HOAT: 1-Hydroxy-7-azabenzotriazole
HOOBT: 3-Hydroxy-1,2,3-benzotriazin-4(3H)-one

### Pd(PPh3)4: Tetrakis(triphenylphosphine)palladium(0)

### SAL: Salicylaldehyde

TFA: Trifluoroacetic acid

### TFE: 2,2,2-Trifluoroethanol

### TIPS: Triisopropylsilane

### Synthesis of Teixobactin Analogues

Teixobactin analogues in Tables 1 and 2 are synthesized through the methods as the aforementioned general procedures. Syntheses of some of the teixobactin analogues are described in details below.

### Example 1

### Synthesis of Chg10-teixobactin

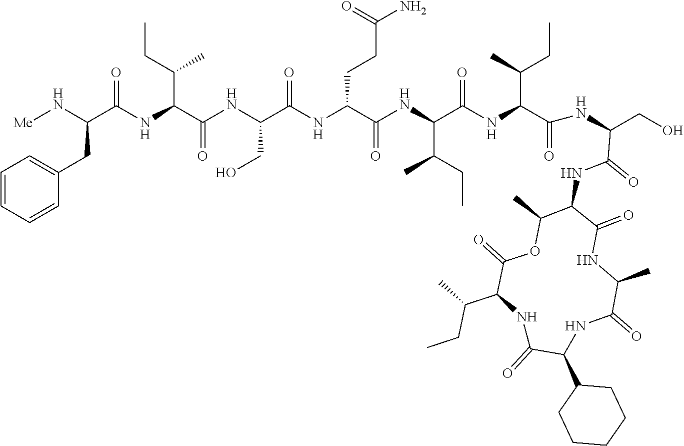 [SMILES: CC[C@@H](C)[C@@H](NC(=O)[C@@H](CCC(N)=O)NC(=O)[C@H](CO)NC(=O)[C@@H](NC(=O)[C@@H](Cc1ccccc1)NC)[C@@H](C)CC)C(=O)N[C@H](C(=O)N[C@@H](CO)C(=O)N[C@H]1C(=O)N[C@@H](C)C(=O)N[C@@H](C2CCCCC2)C(=O)N[C@@H]([C@@H](C)CC)C(=O)O[C@H]1C)[C@@H](C)CC]

Linear peptide resin-Thr[O-Ile-Chg-Ala-NH₂]-Ser(OtBu)NHBoc was synthesized by 9H-fluoren-9-yl-methoxycarbonyl (Fmoc) solid phase peptide synthesis protocol. The peptide was cleaved from the 2-chlorotrityl resin under mild conditions (TFE/AcOH/DCM). After drying, the peptide was cyclized using HATU/HOAt/OxymaPure in CH₂Cl₂ (dichloromethane, DCM) at a concentration of 0.1 mM for 24 h at room temperature. CH₂Cl₂ was evaporated under low pressure and the residue was treated with a mixture of 5 mL TFA/phenol/H₂O (v/v/v=95:2.5:2.5) for 1.5 hours. The mixture of TFA/phenol/H₂O was blown off by a condensed air stream and residue was washed by ethyl ether. The crude compound was dissolved in 1 mL MeOH/HCOOH (v/v=9:1) and hydrogenized by Pd(OH)₂ (10% on carbon) under H₂ (50 atm) for 10 hours. The reaction mixture was filtrated and concentrated in vacuum. The residue was purified by preparative HPLC (5-50% CH₃CN/H₂O over 30 min) to afford the side chain unprotected cyclic peptide.

The linear peptide D-N-Me-Phe-L-Ile-L-Ser-D-Gln-D-allo-Ile-L-Ile-salicylaldehyde ester was synthesized as follows: aminomethyl resin (Chemimpex, loading 1.1 mmol/g, 500 mg) was swollen in anhydrous DCM for 20 min. After DCM was drained, a mixture of 3-(2-acetoxyphenyl)acrylic acid, HATU (O-(7-Azabenzotriazol-1-yl)-NNN′,N′-tetramethyluronium, 2.0 equiv.) and DIEA (N,N-Diisopropylethylamine, 4.0 equiv.) in DMF (N,N-Dimethylformamide) was added. The reaction mixture was stirred for 12 hours at room temperature. The resin was washed with DMF and DCM. Then a solution of 20% piperidine in 5 mL DMF was added to the above resin. The mixture was stirred for 1 hour at room temperature. Then the resin was washed by DCM (5 mL×3) and DMF (5 mL×3). Then, Boc-SPPS (Boc-solid phase peptide synthesis) was performed with Boc-Ile-OH, Boc-D-allo-Ile, D-Gln-OH, Boc-Ser(Bn)-OH, Boc-Ile-OH, and Boc-NMe-D-Phe-OH. Before peptide was cleaved from resin, it was treated with a mixture of TMSOTf (Trimethylsilyl trifluoromethanesulfonate)/TFA(Trifluoroacetic acid)/thioanisole (1:8.5:0.5, v/v/v) at 0° C. for 1 hour to remove the protective groups. Then the resin was washed by DCM, after which the resin was added into DCM/TFA (95:5, v/v) at −78° C. and treated with O₃ for 5 minutes. Me₂S was added and the solution was allowed to warm up and stirred at room temperature for another 1 hour. The reaction mixture was concentrated under low pressure and purified by preparative HPLC (20-60% CH₃CN/H₂O over 30 min) to afford the desired peptide salicylaldehyde ester.

The resulting peptide salicylaldehyde ester and the cyclic peptide were dissolved in a mixture of pyridine/AcOH (mole/mole=1:1) and stirred at room temperature for 10 hours. After the solvent was removed by lyophilization, 1 mL TFA/H₂O/TIPS (v/v/v=94:5:1) was added and stirred for 1 hour. TFA/H₂O/TIPS was blown away by a condensed air stream. The residue was purified by preparative HPLC (20-60% CH₃CN/H₂O over 30 min) to obtained Chg10-teixobactin.

### Example 2

### Synthesis of Cha10-teixobactin

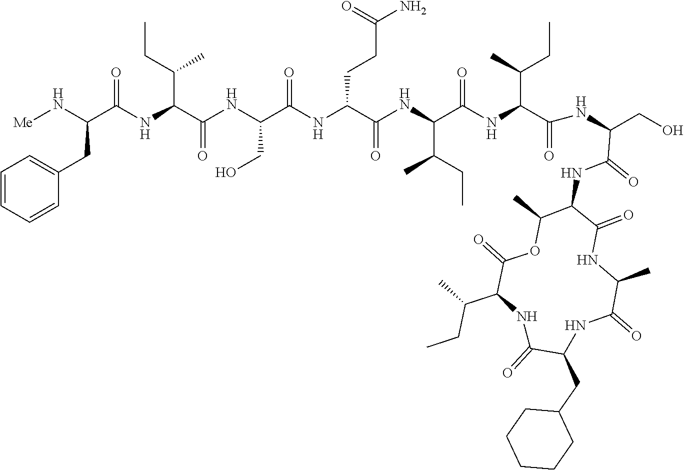 [SMILES: CC[C@@H](C)[C@@H](NC(=O)[C@@H](CCC(N)=O)NC(=O)[C@H](CO)NC(=O)[C@@H](NC(=O)[C@@H](Cc1ccccc1)NC)[C@@H](C)CC)C(=O)N[C@H](C(=O)N[C@@H](CO)C(=O)N[C@H]1C(=O)N[C@@H](C)C(=O)N[C@@H](CC2CCCCC2)C(=O)N[C@@H]([C@@H](C)CC)C(=O)O[C@H]1C)[C@@H](C)CC]

Linear peptide resin-Thr[O-Ile-Cha-Ala-NH₂]-Ser(OtBu)NHBoc was synthesized by 9H-fluoren-9-yl-methoxycarbonyl (Fmoc) solid phase peptide synthesis protocol. The peptide was cleaved from the 2-chlorotrityl resin under mild conditions (TFE/AcOH/DCM). After dryness, the peptide was cyclized using HATU/HOAt/OxymaPure in DCM at a concentration of 0.1 mM for 24 h at room temperature. DCM was evaporated under low pressure and the residue was treated with a mixture of 5 mL TFA/phenol/H₂O (v/v/v=95:2.5:2.5) for 1.5 hours. The mixture of TFA/phenol/H₂O was blown off by a condensed air stream and residue was washed by ethyl ether. The crude compound was dissolved in 1 mL MeOH/HCOOH (v/v=9:1) and hydrogenized by Pd(OH)₂ (10% on carbon) under H₂ (50 atm) for 10 hours. The reaction mixture was filtrated and concentrated in vacuum. The residue was purified by preparative HPLC (5-50% CH₃CN/H₂O over 30 min) to afford the side chain unprotected cyclic peptide.

The linear peptide D-N-Me-Phe-L-Ile-L-Ser-D-Gln-D-allo-Ile-L-Ile-salicylaldehyde ester was synthesized as follows: aminomethyl resin (Chemimpex, loading 1.1 mmol/g, 500 mg) was swollen in anhydrous DCM for 20 min. After DCM was drained, a mixture of compound 3-(2-acetoxyphenyl)acrylic acid, HATU (2.0 equiv.) and DIEA (4.0 equiv.) in DMF was added. The reaction mixture was stirred for 12 hours at room temperature. The resin was washed with DMF and DCM. Then a solution of 20% piperidine in 5 mL DMF was added to the above resin. The mixture was stirred for 1 hour at room temperature. Then the resin was washed by DCM (5 mL×3) and DMF (5 mL×3). Then, Boc-SPPS was performed with Boc-Ile-OH, Boc-D-allo-Ile, D-Gln-OH, Boc-Ser(Bn)-OH, Boc-Ile-OH, and Boc-NMe-D-Phe-OH. Before peptide was cleaved from resin, it was treated with a mixture of TMSOTf/TFA/thioanisole (1:8.5:0.5, v/v/v) at 0° C. for 1 hour to remove the protective groups. Then the resin was washed by DCM, after which the resin was added into DCM/TFA (95:5, v/v) at −78° C. and treated with O₃ for 5 min. Following the addition of Me₂S, the solution was allowed to warm up and stirred at room temperature for another 1 hour. The reaction mixture was concentrated under low pressure and purified by preparative HPLC (20-60% CH₃CN/H₂O over 30 min) to obtain the desired peptide salicylaldehyde ester.

The resultant peptide salicylaldehyde ester and the cyclic peptide were dissolved in a mixture of pyridine/AcOH (mole/mole=1:1) and stirred at room temperature for 10 hours. After the solvent was removed by lyophilization, 1 mL TFA/H₂O/TIPS (v/v/v=94:5:1) was added and stirred for 1 hour. TFA/H₂O/TIPS was blown away by a condensed air stream. The residue was purified by preparative HPLC (20-60% CH₃CN/H₂O over 30 min) to obtain the resultant Cha10-teixobactin.

### Example 3

### Synthesis of β-cyclopropyl-Ala10-teixobactin

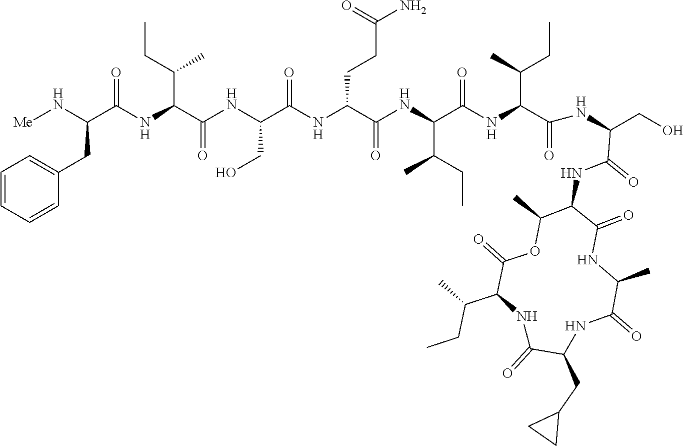 [SMILES: CC[C@@H](C)[C@@H](NC(=O)[C@@H](CCC(N)=O)NC(=O)[C@H](CO)NC(=O)[C@@H](NC(=O)[C@@H](Cc1ccccc1)NC)[C@@H](C)CC)C(=O)N[C@H](C(=O)N[C@@H](CO)C(=O)N[C@H]1C(=O)N[C@@H](C)C(=O)N[C@@H](CC2CC2)C(=O)N[C@@H]([C@@H](C)CC)C(=O)O[C@H]1C)[C@@H](C)CC]

Linear peptide resin-Thr[O-Ile-(cyclopropyl-Ala)-Ala-NH₂]-Ser(OtBu)NHBoc was synthesized by 9H-fluoren-9-yl-methoxycarbonyl (Fmoc) solid phase peptide synthesis protocol. The peptide was cleaved from the 2-chlorotrityl resin under mild conditions (TFE/AcOH/DCM). After dryness, the peptide was cyclized using HATU/HOAt/OxymaPure in DCM at a concentration of 0.1 mM for 24 h at room temperature. DCM was evaporated under low pressure and the residue was treated with a mixture of 5 mL TFA/phenol/H₂O (v/v/v=95:2.5:2.5) for 1.5 hours. The mixture of TFA/phenol/H₂O was blown off by a condensed air stream and residue was washed by ethyl ether. The crude compound was dissolved in 1 mL MeOH/HCOOH (v/v=9:1) and hydrogenized by Pd(OH)₂ (10% on carbon) under H₂ (50 atm) for 10 hours. The reaction mixture was filtrated and concentrated in vacuum. The residue was purified by preparative HPLC (5-50% CH₃CN/H₂O over 30 min) to afford the side chain unprotected cyclic peptide.

The linear peptide D-N-Me-Phe-L-Ile-L-Ser-D-Gln-D-allo-Ile-L-Ile-salicylaldehyde ester was synthesized as follows: aminomethyl resin (Chemimpex, loading 1.1 mmol/g, 500 mg) was swollen in anhydrous DCM for 20 min. After DCM was drained, a mixture of 3-(2-acetoxyphenyl)acrylic acid, HATU (2.0 equiv.) and DIEA (4.0 equiv.) in DMF was added. The reaction mixture was stirred for 12 hours at room temperature. The resin was washed with DMF and DCM. Then a solution of 20% piperidine in 5 mL DMF was added to the above resin. The mixture was stirred for 1 hour at room temperature. Then the resin was washed by DCM (5 mL×3) and DMF (5 mL×3). Then, Boc-SPPS was performed with Boc-Ile-OH, Boc-D-allo-Ile, D-Gln-OH, Boc-Ser(Bn)-OH, Boc-Ile-OH, and Boc-NMe-D-Phe-OH. Before the peptide was cleaved from resin, it was treated with a mixture of TMSOTf/TFA/thioanisole (1:8.5:0.5, v/v/v) at 0° C. for 1 hour to remove the protective groups. Then the resin was washed by DCM, after which the resin was added into DCM/TFA (95:5, v/v) at −78° C. and treated with O₃ for 5 min. Following the addition of Me₂S, the solution was allowed to warm up and stirred at room temperature for another 1 hour. The reaction mixture was concentrated under low pressure and purified by preparative HPLC (20-60% CH₃CN/H₂O over 30 min) to afford the desired peptide salicylaldehyde ester.

The resultant peptide salicylaldehyde ester and the cyclic peptide were dissolved in a mixture of pyridine/AcOH (mole/mole=1:1) and stirred at room temperature for 10 hours. After the solvent was removed by lyophilization, 1 mL TFA/H₂O/TIPS (v/v/v=94:5:1) was added and stirred for 1 hour. TFA/H₂O/TIPS was blown away by a condensed air stream. The residue was purified by preparative HPLC (20-60% CH₃CN/H₂O over 30 min) to obtain β-cyclopropyl-Ala10-teixobactin.

### Example 4

### Synthesis of Nle10-teixobactin

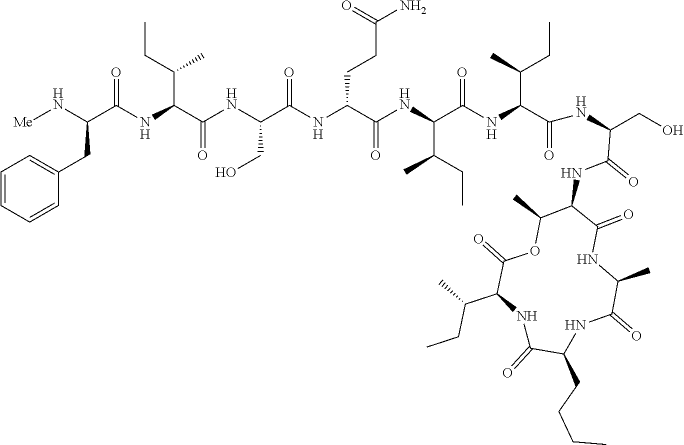 [SMILES: CCCC[C@@H]1NC(=O)[C@H](C)NC(=O)[C@H](NC(=O)[C@H](CO)NC(=O)[C@@H](NC(=O)[C@H](NC(=O)[C@@H](CCC(N)=O)NC(=O)[C@H](CO)NC(=O)[C@@H](NC(=O)[C@@H](Cc2ccccc2)NC)[C@@H](C)CC)[C@H](C)CC)[C@@H](C)CC)[C@H](C)OC(=O)[C@H]([C@@H](C)CC)NC1=O]

Linear peptide resin-Thr[O-Ile-Nle-Ala-NH₂]-Ser(OtBu)NHBoc was synthesized by 9H-fluoren-9-yl-methoxycarbonyl (Fmoc) solid phase peptide synthesis protocol. The peptide was cleaved from the 2-chlorotrityl resin under mild conditions (TFE/AcOH/DCM). After dryness, the peptide was cyclized using HATU/HOAt/OxymaPure in DCM at a concentration of 0.1 mM for 24 h at room temperature. DCM was evaporated under low pressure and the residue was treated with a mixture of 5 mL TFA/phenol/H₂O (v/v/v=95:2.5:2.5) for 1.5 hours. The mixture of TFA/phenol/H₂O was blown off by a condensed air stream and residue was washed by ethyl ether. The crude compound was dissolved in 1 mL MeOH/HCOOH (v/v=9:1) and hydrogenized by Pd(OH)₂ (10% on carbon) under H₂ (50 atm) for 10 hours. The reaction mixture was filtered and concentrated in vacuum. The residue was purified by preparative HPLC (5-50% CH₃CN/H₂O over 30 min) to afford the side chain unprotected cyclic peptide.

The linear peptide D-N-Me-Phe-L-Ile-L-Ser-D-Gln-D-allo-Ile-L-Ile-salicylaldehyde ester was synthesized as follows: aminomethyl resin (Chemimpex, loading 1.1 mmol/g, 500 mg) was swollen in anhydrous DCM for 20 min. After DCM was drained, a mixture of 3-(2-acetoxyphenyl)acrylic acid, HATU (2.0 equiv.) and DIEA (4.0 equiv.) in DMF was added. The reaction mixture was stirred for 12 hours at room temperature. The resin was washed with DMF and DCM. Then a solution of 20% piperidine in 5 mL DMF was added to the above resin. The mixture was stirred for 1 hour at room temperature. Then the resin was washed by DCM (5 mL×3) and DMF (5 mL×3). Then, Boc-SPPS was performed with Boc-Ile-OH, Boc-D-allo-Ile, D-Gln-OH, Boc-Ser(Bn)-OH, Boc-Ile-OH, and Boc-NMe-D-Phe-OH. Before peptide was cleaved from resin, it was treated with a mixture of TMSOTf/TFA/thioanisole (1:8.5:0.5, v/v/v) at 0° C. for 1 hour to remove the protective groups. Then the resin was washed by DCM, after which the resin was added into DCM/TFA (95:5, v/v) at −78° C. and treated with O₃ for 5 min. Following the addition of Me₂S, the solution was allowed to warm up and stirred at room temperature for another 1 hour. The reaction mixture was concentrated under low pressure and purified by preparative HPLC (20-60% CH₃CN/H₂O over 30 min) to afford the desired peptide salicylaldehyde ester.

The obtained peptide salicylaldehyde ester and the cyclic peptide were dissolved in a mixture of pyridine/AcOH (mole/mole=1:1) and stirred at room temperature for 10 hours. After the solvent was removed by lyophilization, 1 mL TFA/H₂O/TIPS (v/v/v=94:5:1) was added and stirred for 1 hour. TFA/H₂O/TIPS was blown away by a condensed air stream. The residue was purified by preparative HPLC (20-60% CH₃CN/H₂O over 30 min) to afford Nle10-teixobactin.

### Example 5

### Synthesis of Met10-teixobactin/methibactin

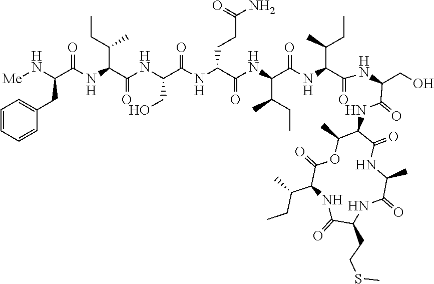 [SMILES: CC[C@@H](C)[C@@H](NC(=O)[C@@H](CCC(N)=O)NC(=O)[C@H](CO)NC(=O)[C@@H](NC(=O)[C@@H](Cc1ccccc1)NC)[C@@H](C)CC)C(=O)N[C@H](C(=O)N[C@@H](CO)C(=O)N[C@H]1C(=O)N[C@@H](C)C(=O)N[C@@H](CCSC)C(=O)N[C@@H]([C@@H](C)CC)C(=O)O[C@H]1C)[C@@H](C)CC]

Linear peptide resin-Thr[O-Ile-Met-Ala-NH₂]-Ser(OtBu)NHBoc was synthesized by 9H-fluoren-9-yl-methoxycarbonyl (Fmoc) solid phase peptide synthesis protocol. The peptide was cleaved from the 2-chlorotrityl resin under the mild condition (TFE/AcOH/DCM). After dryness, the peptide was cyclized using HATU/HOAt/OxymaPure in CH₂Cl₂ at a concentration of 0.1 mM for 24 h at room temperature. CH₂Cl₂ was evaporated under low pressure and the residue was treated with a mixture of 5 mL TFA/phenol/H₂O (v/v/v=95:2.5:2.5) for 1.5 hours. The mixture of TFA/phenol/H₂O was blown off by a condensed air stream and residue was washed by ethyl ether. The crude compound was dissolved in 1 mL MeOH/HCOOH (v/v=9:1) and hydrogenized by Pd(OH)₂ (10% on carbon) under H₂ (50 atm) for 10 hours. The reaction mixture was filtrated and concentrated in vacuo. The residue was purified by preparative HPLC (5-50% CH₃CN/H₂O over 30 min) to afford the side chain unprotected cyclic peptide.

The linear peptide D-N-Me-Phe-L-Ile-L-Ser-D-Gln-D-allo-Ile-L-Ile-salicylaldehyde ester was synthesized as follows: aminomethyl resin (Chemimpex, loading 1.1 mmol/g, 500 mg) was swollen in anhydrous DCM for 20 min. After DCM was drained, a mixture of compound 3-(2-acetoxyphenyl)acrylic acid, HATU (2.0 equiv.) and DIEA (4.0 equiv.) in DMF was added. The reaction mixture was shaken for 12 hours at room temperature. The resin was washed with DMF and DCM. Then a solution of 20% piperidine in 5 mL DMF was added to the above resin. The mixture was shaken for 1 hour at room temperature. Then the resin was washed by DCM (5 mL×3) and DMF (5 mL×3). Then, Boc-SPPS was performed with Boc-Ile-OH, Boc-D-allo-Ile, D-Gln-OH, Boc-Ser(Bn)-OH, Boc-Ile-OH, and Boc-NMe-D-Phe-OH. Before peptide was cleaved from resin, it was treated with a mixture of TMSOTf/TFA/thioanisole (1:8.5:0.5, v/v/v) at 0° C. for 1 hour to remove the protective groups. Then the resin was washed by DCM, after which the resin was added into DCM/TFA (95:5, v/v) at −78° C. and treated with O₃ for 5 min. Following the addition of Me₂S, the solution was allowed to warm up and stirred at room temperature for another 1 hour. The reaction mixture was concentrated under low pressure and purified by preparative HPLC (20-60% CH₃CN/H₂O over 30 min) to afford the desired peptide salicylaldehyde ester.

The obtained peptide salicylaldehyde ester and the cyclic peptide were dissolved in a mixture of pyridine/AcOH (mole/mole=1:1) and stirred at room temperature for 10 hours. After the solvent was removed by lyophilization, 1 mL TFA/H₂O/TIPS (v/v/v=94:5:1) was added and stirred for 1 hour. TFA/H₂O/TIPS was blown away by a condensed air stream. The residue was purified by preparative HPLC (20-60% CH₃CN/H₂O over 30 min) to afford Met10-teixobactin/Methibactin.

### Example 6

### Synthesis of T83

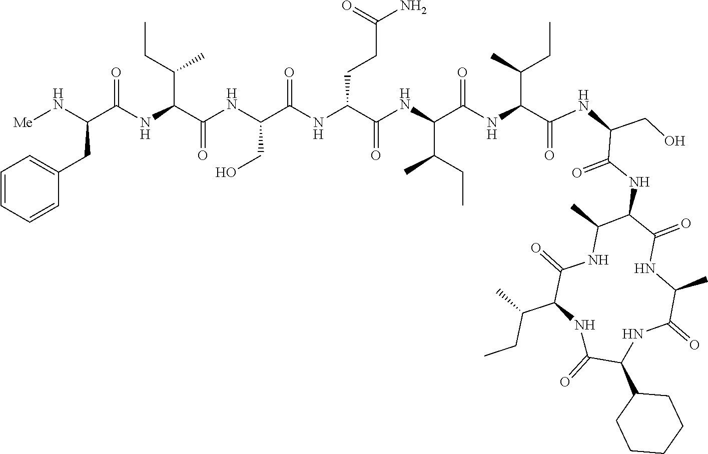 [SMILES: CC[C@@H](C)[C@@H](NC(=O)[C@@H](CCC(N)=O)NC(=O)[C@H](CO)NC(=O)[C@@H](NC(=O)[C@@H](Cc1ccccc1)NC)[C@@H](C)CC)C(=O)N[C@H](C(=O)N[C@@H](CO)C(=O)N[C@H]1C(=O)N[C@@H](C)C(=O)N[C@@H](C2CCCCC2)C(=O)N[C@@H]([C@@H](C)CC)C(=O)N[C@H]1C)[C@@H](C)CC]

Linear peptide resin-Thr[NH-Ile-Chg-Ala-NH₂]-Ser(OtBu)NHBoc was synthesized by 9H-fluoren-9-yl-methoxycarbonyl (Fmoc) solid phase peptide synthesis protocol. The peptide was cleaved from the 2-chlorotrityl resin under the mild condition (TFE/AcOH/DCM). After dryness, the peptide was cyclized using HATU/HOAt/OxymaPure in CH₂Cl₂ at a concentration of 0.1 mM for 24 h at room temperature. CH₂Cl₂ was evaporated under low pressure and the residue was treated with a mixture of 5 mL TFA/phenol/H₂O (v/v/v=95:2.5:2.5) for 1.5 hours. The mixture of TFA/phenol/H₂O was blown off by a condensed air stream and residue was washed by ethyl ether. The residue was purified by preparative HPLC (5-50% CH₃CN/H₂O over 30 min) to afford the side chain unprotected cyclic peptide.

The linear peptide D-N-Me-Phe-L-Ile-L-Ser-D-Gln-D-allo-Ile-L-Ile-salicylaldehyde ester was synthesized as follows: aminomethyl resin (Chemimpex, loading 1.1 mmol/g, 500 mg) was swollen in anhydrous DCM for 20 min. After DCM was drained, a mixture of compound 3-(2-acetoxyphenyl)acrylic acid, HATU (2.0 equiv.) and DIEA (4.0 equiv.) in DMF was added. The reaction mixture was shaken for 12 hours at room temperature. The resin was washed with DMF and DCM. Then a solution of 20% piperidine in 5 mL DMF was added to the above resin. The mixture was shaken for 1 hour at room temperature. Then the resin was washed by DCM (5 mL×3) and DMF (5 mL×3). Then, Boc-SPPS was performed with Boc-Ile-OH, Boc-D-allo-Ile, D-Gln-OH, Boc-Ser(Bn)-OH, Boc-Ile-OH, and Boc-NMe-D-Phe-OH. Before peptide was cleaved from resin, it was treated with a mixture of TMSOTf/TFA/thioanisole (1:8.5:0.5, v/v/v) at 0° C. for 1 hour to remove the protective groups. Then the resin was washed by DCM, after which the resin was added into DCM/TFA (95:5, v/v) at −78° C. and treated with O₃ for 5 min. Following the addition of Me₂S, the solution was allowed to warm up and stirred at room temperature for another 1 hour. The reaction mixture was concentrated under low pressure and purified by preparative HPLC (20-60% CH₃CN/H₂O over 30 min) to afford the desired peptide salicylaldehyde ester.

The obtained peptide salicylaldehyde ester and the cyclic peptide were dissolved in a mixture of pyridine/AcOH (mole/mole=1:1) and stirred at room temperature for 10 hours. After the solvent was removed by lyophilization, 1 mL TFA/H₂O/TIPS (v/v/v=94:5:1) was added and stirred for 1 hour. TFA/H₂O/TIPS was blown away by a condensed air stream. The residue was purified by preparative HPLC (20-60% CH₃CN/H₂O over 30 min) to afford T83.

### Example 7

### Synthesis of T84

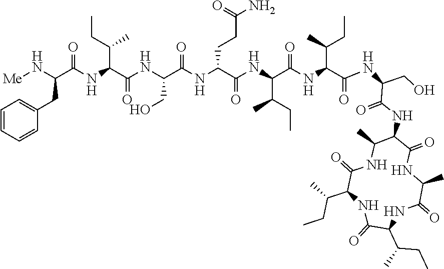 [SMILES: CC[C@@H](C)[C@@H](NC(=O)[C@@H](CCC(N)=O)NC(=O)[C@H](CO)NC(=O)[C@@H](NC(=O)[C@@H](Cc1ccccc1)NC)[C@@H](C)CC)C(=O)N[C@H](C(=O)N[C@@H](CO)C(=O)N[C@H]1C(=O)N[C@@H](C)C(=O)N[C@@H]([C@@H](C)CC)C(=O)N[C@@H]([C@@H](C)CC)C(=O)N[C@H]1C)[C@@H](C)CC]

Linear peptide resin-Thr[NH-Ile-Ile-Ala-NH₂]-Ser(OtBu)NHBoc was synthesized by 9H-fluoren-9-yl-methoxycarbonyl (Fmoc) solid phase peptide synthesis protocol. The peptide was cleaved from the 2-chlorotrityl resin under the mild condition (TFE/AcOH/DCM). After dryness, the peptide was cyclized using HATU/HOAt/OxymaPure in CH₂Cl₂ at a concentration of 0.1 mM for 24 h at room temperature. CH₂Cl₂ was evaporated under low pressure and the residue was treated with a mixture of 5 mL TFA/phenol/H₂O (v/v/v=95:2.5:2.5) for 1.5 hours. The mixture of TFA/phenol/H₂O was blown off by a condensed air stream and residue was washed by ethyl ether. The residue was purified by preparative HPLC (5-50% CH₃CN/H₂O over 30 min) to afford the side chain unprotected cyclic peptide.

The linear peptide D-N-Me-Phe-L-Ile-L-Ser-D-Gln-D-allo-Ile-L-Ile-salicylaldehyde ester was synthesized as follows: aminomethyl resin (Chemimpex, loading 1.1 mmol/g, 500 mg) was swollen in anhydrous DCM for 20 min. After DCM was drained, a mixture of compound 3-(2-acetoxyphenyl)acrylic acid, HATU (2.0 equiv.) and DIEA (4.0 equiv.) in DMF was added. The reaction mixture was shaken for 12 hours at room temperature. The resin was washed with DMF and DCM. Then a solution of 20% piperidine in 5 mL DMF was added to the above resin. The mixture was shaken for 1 hour at room temperature. Then the resin was washed by DCM (5 mL×3) and DMF (5 mL×3). Then, Boc-SPPS was performed with Boc-Ile-OH, Boc-D-allo-Ile, D-Gln-OH, Boc-Ser(Bn)-OH, Boc-Ile-OH, and Boc-NMe-D-Phe-OH. Before peptide was cleaved from resin, it was treated with a mixture of TMSOTf/TFA/thioanisole (1:8.5:0.5, v/v/v) at 0° C. for 1 hour to remove the protective groups. Then the resin was washed by DCM, after which the resin was added into DCM/TFA (95:5, v/v) at −78° C. and treated with O₃ for 5 min. Following the addition of Me₂S, the solution was allowed to warm up and stirred at room temperature for another 1 hour. The reaction mixture was concentrated under low pressure and purified by preparative HPLC (20-60% CH₃CN/H₂O over 30 min) to afford the desired peptide salicylaldehyde ester.

The obtained peptide salicylaldehyde ester and the cyclic peptide were dissolved in a mixture of pyridine/AcOH (mole/mole=1:1) and stirred at room temperature for 10 hours. After the solvent was removed by lyophilization, 1 mL TFA/H₂O/TIPS (v/v/v=94:5:1) was added and stirred for 1 hour. TFA/H₂O/TIPS was blown away by a condensed air stream. The residue was purified by preparative HPLC (20-60% CH₃CN/H₂O over 30 min) to afford T84.

### Example 8

### Synthesis of T85

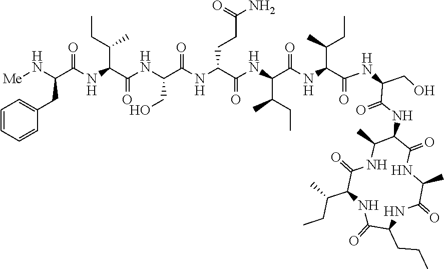 [SMILES: CCC[C@@H]1NC(=O)[C@H](C)NC(=O)[C@H](NC(=O)[C@H](CO)NC(=O)[C@@H](NC(=O)[C@H](NC(=O)[C@@H](CCC(N)=O)NC(=O)[C@H](CO)NC(=O)[C@@H](NC(=O)[C@@H](Cc2ccccc2)NC)[C@@H](C)CC)[C@H](C)CC)[C@@H](C)CC)[C@H](C)NC(=O)[C@H]([C@@H](C)CC)NC1=O]

Linear peptide resin-Thr[NH-Ile-Nva-Ala-NH₂]-Ser(OtBu)NHBoc was synthesized by 9H-fluoren-9-yl-methoxycarbonyl (Fmoc) solid phase peptide synthesis protocol. The peptide was cleaved from the 2-chlorotrityl resin under the mild condition (TFE/AcOH/DCM). After dryness, the peptide was cyclized using HATU/HOAt/OxymaPure in CH₂Cl₂ at a concentration of 0.1 mM for 24 h at room temperature. CH₂Cl₂ was evaporated under low pressure and the residue was treated with a mixture of 5 mL TFA/phenol/H₂O (v/v/v=95:2.5:2.5) for 1.5 hours. The mixture of TFA/phenol/H₂O was blown off by a condensed air stream and residue was washed by ethyl ether. The residue was purified by preparative HPLC (5-50% CH₃CN/H₂O over 30 min) to afford the side chain unprotected cyclic peptide.

The linear peptide D-N-Me-Phe-L-Ile-L-Ser-D-Gln-D-allo-Ile-L-Ile-salicylaldehyde ester was synthesized as follows: aminomethyl resin (Chemimpex, loading 1.1 mmol/g, 500 mg) was swollen in anhydrous DCM for 20 min. After DCM was drained, a mixture of compound 3-(2-acetoxyphenyl)acrylic acid, HATU (2.0 equiv.) and DIEA (4.0 equiv.) in DMF was added.

The reaction mixture was shaken for 12 hours at room temperature. The resin was washed with DMF and DCM. Then a solution of 20% piperidine in 5 mL DMF was added to the above resin. The mixture was shaken for 1 hour at room temperature. Then the resin was washed by DCM (5 mL×3) and DMF (5 mL×3). Then, Boc-SPPS was performed with Boc-Ile-OH, Boc-D-allo-Ile, D-Gln-OH, Boc-Ser(Bn)-OH, Boc-Ile-OH, and Boc-NMe-D-Phe-OH. Before peptide was cleaved from resin, it was treated with a mixture of TMSOTf/TFA/thioanisole (1:8.5:0.5, v/v/v) at 0° C. for 1 hour to remove the protective groups. Then the resin was washed by DCM, after which the resin was added into DCM/TFA (95:5, v/v) at −78° C. and treated with O₃ for 5 min. Following the addition of Me₂S, the solution was allowed to warm up and stirred at room temperature for another 1 hour. The reaction mixture was concentrated under low pressure and purified by preparative HPLC (20-60% CH₃CN/H₂O over 30 min) to afford the desired peptide salicylaldehyde ester.

The obtained peptide salicylaldehyde ester and the cyclic peptide were dissolved in a mixture of pyridine/AcOH (mole/mole=1:1) and stirred at room temperature for 10 hours. After the solvent was removed by lyophilization, 1 mL TFA/H₂O/TIPS (v/v/v=94:5:1) was added and stirred for 1 hour. TFA/H₂O/TIPS was blown away by a condensed air stream. The residue was purified by preparative HPLC (20-60% CH₃CN/H₂O over 30 min) to afford T85.

### Example 9

### Synthesis of T86

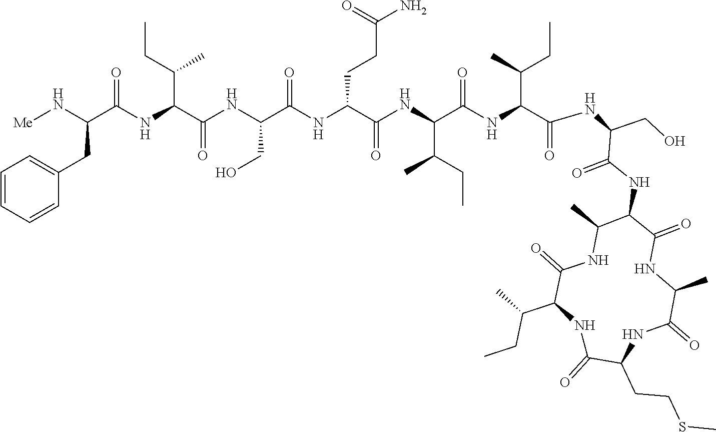 [SMILES: CC[C@@H](C)[C@@H](NC(=O)[C@@H](CCC(N)=O)NC(=O)[C@H](CO)NC(=O)[C@@H](NC(=O)[C@@H](Cc1ccccc1)NC)[C@@H](C)CC)C(=O)N[C@H](C(=O)N[C@@H](CO)C(=O)N[C@H]1C(=O)N[C@@H](C)C(=O)N[C@@H](CCSC)C(=O)N[C@@H]([C@@H](C)CC)C(=O)N[C@H]1C)[C@@H](C)CC]

Linear peptide resin-Thr[NH-Ile-Met-Ala-NH₂]-Ser(OtBu)NHBoc was synthesized by 9H-fluoren-9-yl-methoxycarbonyl (Fmoc) solid phase peptide synthesis protocol. The peptide was cleaved from the 2-chlorotrityl resin under the mild condition (TFE/AcOH/DCM). After dryness, the peptide was cyclized using HATU/HOAt/OxymaPure in CH₂Cl₂ at a concentration of 0.1 mM for 24 h at room temperature. CH₂Cl₂ was evaporated under low pressure and the residue was treated with a mixture of 5 mL TFA/phenol/H₂O (v/v/v=95:2.5:2.5) for 1.5 hours. The mixture of TFA/phenol/H₂O was blown off by a condensed air stream and residue was washed by ethyl ether. The residue was purified by preparative HPLC (5-50% CH₃CN/H₂O over 30 min) to afford the side chain unprotected cyclic peptide.

The linear peptide D-N-Me-Phe-L-Ile-L-Ser-D-Gln-D-allo-Ile-L-Ile-salicylaldehyde ester was synthesized as follows: aminomethyl resin (Chemimpex, loading 1.1 mmol/g, 500 mg) was swollen in anhydrous DCM for 20 min. After DCM was drained, a mixture of compound 3-(2-acetoxyphenyl)acrylic acid, HATU (2.0 equiv.) and DIEA (4.0 equiv.) in DMF was added. The reaction mixture was shaken for 12 hours at room temperature. The resin was washed with DMF and DCM. Then a solution of 20% piperidine in 5 mL DMF was added to the above resin. The mixture was shaken for 1 hour at room temperature. Then the resin was washed by DCM (5 mL×3) and DMF (5 mL×3). Then, Boc-SPPS was performed with Boc-Ile-OH, Boc-D-allo-Ile, D-Gln-OH, Boc-Ser(Bn)-OH, Boc-Ile-OH, and Boc-NMe-D-Phe-OH. Before peptide was cleaved from resin, it was treated with a mixture of TMSOTf/TFA/thioanisole (1:8.5:0.5, v/v/v) at 0° C. for 1 hour to remove the protective groups. Then the resin was washed by DCM, after which the resin was added into DCM/TFA (95:5, v/v) at −78° C. and treated with O₃ for 5 min. Following the addition of Me₂S, the solution was allowed to warm up and stirred at room temperature for another 1 hour. The reaction mixture was concentrated under low pressure and purified by preparative HPLC (20-60% CH₃CN/H₂O over 30 min) to afford the desired peptide salicylaldehyde ester.

The obtained peptide salicylaldehyde ester and the cyclic peptide were dissolved in a mixture of pyridine/AcOH (mole/mole=1:1) and stirred at room temperature for 10 hours. After the solvent was removed by lyophilization, 1 mL TFA/H₂O/TIPS (v/v/v=94:5:1) was added and stirred for 1 hour. TFA/H₂O/TIPS was blown away by a condensed air stream. The residue was purified by preparative HPLC (20-60% CH₃CN/H₂O over 30 min) to afford T86.

### Example 10

### Synthesis of T87

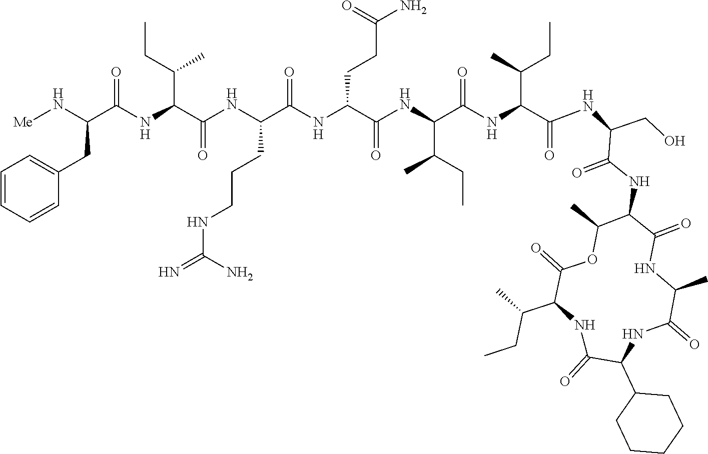 [SMILES: CC[C@@H](C)[C@@H](NC(=O)[C@@H](CCC(N)=O)NC(=O)[C@H](CCCNC(=N)N)NC(=O)[C@@H](NC(=O)[C@@H](Cc1ccccc1)NC)[C@@H](C)CC)C(=O)N[C@H](C(=O)N[C@@H](CO)C(=O)N[C@H]1C(=O)N[C@@H](C)C(=O)N[C@@H](C2CCCCC2)C(=O)N[C@@H]([C@@H](C)CC)C(=O)O[C@H]1C)[C@@H](C)CC]

Linear peptide resin-Thr[O-Ile-Chg-Ala-NH₂]-Ser(OtBu)NHBoc was synthesized by 9H-fluoren-9-yl-methoxycarbonyl (Fmoc) solid phase peptide synthesis protocol. The peptide was cleaved from the 2-chlorotrityl resin under the mild condition (TFE/AcOH/DCM). After dryness, the peptide was cyclized using HATU/HOAt/OxymaPure in CH₂Cl₂ at a concentration of 0.1 mM for 24 h at room temperature. CH₂Cl₂ was evaporated under low pressure and the residue was treated with a mixture of 5 mL TFA/phenol/H₂O (v/v/v=95:2.5:2.5) for 1.5 hours. The mixture of TFA/phenol/H₂O was blown off by a condensed air stream and residue was washed by ethyl ether. The residue was purified by preparative HPLC (5-50% CH₃CN/H₂O over 30 min) to afford the side chain unprotected cyclic peptide.

The linear peptide D-N-Me-Phe-L-Ile-L-Arg-D-Gln-D-allo-Ile-L-Ile-salicylaldehyde ester was synthesized as follows: aminomethyl resin (Chemimpex, loading 1.1 mmol/g, 500 mg) was swollen in anhydrous DCM for 20 min. After DCM was drained, a mixture of compound 3-(2-acetoxyphenyl)acrylic acid, HATU (2.0 equiv.) and DIEA (4.0 equiv.) in DMF was added. The reaction mixture was shaken for 12 hours at room temperature. The resin was washed with DMF and DCM. Then a solution of 20% piperidine in 5 mL DMF was added to the above resin. The mixture was shaken for 1 hour at room temperature. Then the resin was washed by DCM (5 mL×3) and DMF (5 mL×3). Then, Boc-SPPS was performed with Boc-Ile-OH, Boc-D-allo-Ile, D-Gln-OH, Boc-Ser(Bn)-OH, Boc-Ile-OH, and Boc-NMe-D-Phe-OH. Before peptide was cleaved from resin, it was treated with a mixture of TMSOTf/TFA/thioanisole (1:8.5:0.5, v/v/v) at 0° C. for 1 hour to remove the protective groups. Then the resin was washed by DCM, after which the resin was added into DCM/TFA (95:5, v/v) at −78° C. and treated with O₃ for 5 min. Following the addition of Me₂S, the solution was allowed to warm up and stirred at room temperature for another 1 hour. The reaction mixture was concentrated under low pressure and purified by preparative HPLC (20-60% CH₃CN/H₂O over 30 min) to afford the desired peptide salicylaldehyde ester.

The obtained peptide salicylaldehyde ester and the cyclic peptide were dissolved in a mixture of pyridine/AcOH (mole/mole=1:1) and stirred at room temperature for 10 hours. After the solvent was removed by lyophilization, 1 mL TFA/H₂O/TIPS (v/v/v=94:5:1) was added and stirred for 1 hour. TFA/H₂O/TIPS was blown away by a condensed air stream. The residue was purified by preparative HPLC (20-60% CH₃CN/H₂O over 30 min) to afford T87.

### Example 11

### Synthesis of T88

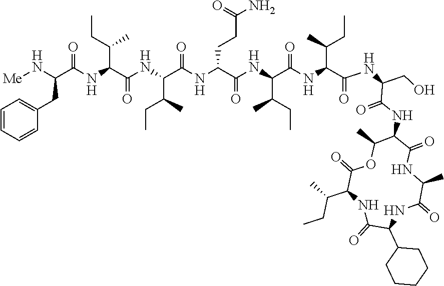 [SMILES: CC[C@@H](C)[C@@H](NC(=O)[C@@H](CCC(N)=O)NC(=O)[C@@H](NC(=O)[C@@H](NC(=O)[C@@H](Cc1ccccc1)NC)[C@@H](C)CC)[C@@H](C)CC)C(=O)N[C@H](C(=O)N[C@@H](CO)C(=O)N[C@H]1C(=O)N[C@@H](C)C(=O)N[C@@H](C2CCCCC2)C(=O)N[C@@H]([C@@H](C)CC)C(=O)O[C@H]1C)[C@@H](C)CC]

Linear peptide resin-Thr[O-Ile-Chg-Ala-NH₂]-Ser(OtBu)NHBoc was synthesized by 9H-fluoren-9-yl-methoxycarbonyl (Fmoc) solid phase peptide synthesis protocol. The peptide was cleaved from the 2-chlorotrityl resin under the mild condition (TFE/AcOH/DCM). After dryness, the peptide was cyclized using HATU/HOAt/OxymaPure in CH₂Cl₂ at a concentration of 0.1 mM for 24 h at room temperature. CH₂Cl₂ was evaporated under low pressure and the residue was treated with a mixture of 5 mL TFA/phenol/H₂O (v/v/v=95:2.5:2.5) for 1.5 hours. The mixture of TFA/phenol/H₂O was blown off by a condensed air stream and residue was washed by ethyl ether. The residue was purified by preparative HPLC (5-50% CH₃CN/H₂O over 30 min) to afford the side chain unprotected cyclic peptide.

The linear peptide D-N-Me-Phe-L-Ile-L-Ile-D-Gln-D-allo-Ile-L-Ile-salicylaldehyde ester was synthesized as follows: aminomethyl resin (Chemimpex, loading 1.1 mmol/g, 500 mg) was swollen in anhydrous DCM for 20 min. After DCM was drained, a mixture of compound 3-(2-acetoxyphenyl)acrylic acid, HATU (2.0 equiv.) and DIEA (4.0 equiv.) in DMF was added. The reaction mixture was shaken for 12 hours at room temperature. The resin was washed with DMF and DCM. Then a solution of 20% piperidine in 5 mL DMF was added to the above resin. The mixture was shaken for 1 hour at room temperature. Then the resin was washed by DCM (5 mL×3) and DMF (5 mL×3). Then, Boc-SPPS was performed with Boc-Ile-OH, Boc-D-allo-Ile, D-Gln-OH, Boc-Ser(Bn)-OH, Boc-Ile-OH, and Boc-NMe-D-Phe-OH. Before peptide was cleaved from resin, it was treated with a mixture of TMSOTf/TFA/thioanisole (1:8.5:0.5, v/v/v) at 0° C. for 1 hour to remove the protective groups. Then the resin was washed by DCM, after which the resin was added into DCM/TFA (95:5, v/v) at −78° C. and treated with O₃ for 5 min. Following the addition of Me₂S, the solution was allowed to warm up and stirred at room temperature for another 1 hour. The reaction mixture was concentrated under low pressure and purified by preparative HPLC (20-60% CH₃CN/H₂O over 30 min) to afford the desired peptide salicylaldehyde ester.

The obtained peptide salicylaldehyde ester and the cyclic peptide were dissolved in a mixture of pyridine/AcOH (mole/mole=1:1) and stirred at room temperature for 10 hours. After the solvent was removed by lyophilization, 1 mL TFA/H₂O/TIPS (v/v/v=94:5:1) was added and stirred for 1 hour. TFA/H₂O/TIPS was blown away by a condensed air stream. The residue was purified by preparative HPLC (20-60% CH₃CN/H₂O over 30 min) to afford T88.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

1. An antibiotic of Formula I or I′, II or II′, III or III′, IV or IV′, V or V′ or VI or VI′, or a salt thereof:

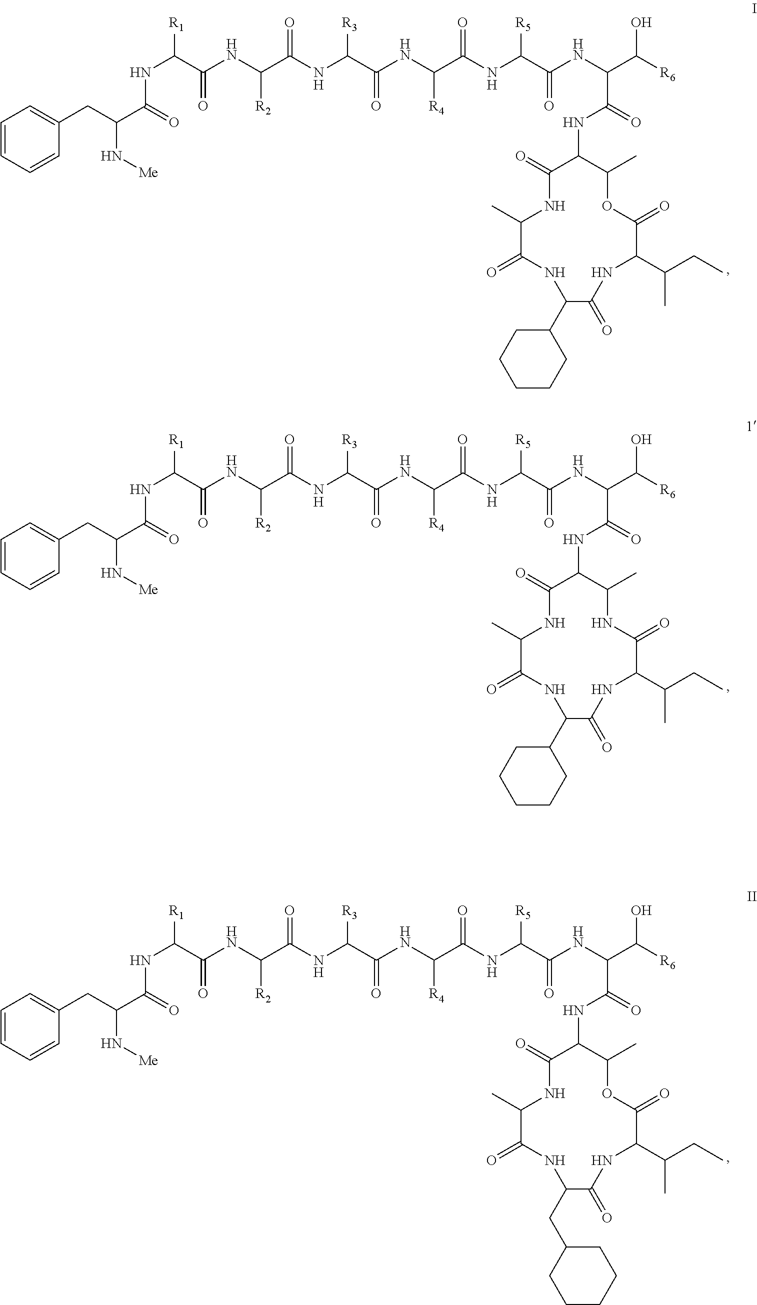

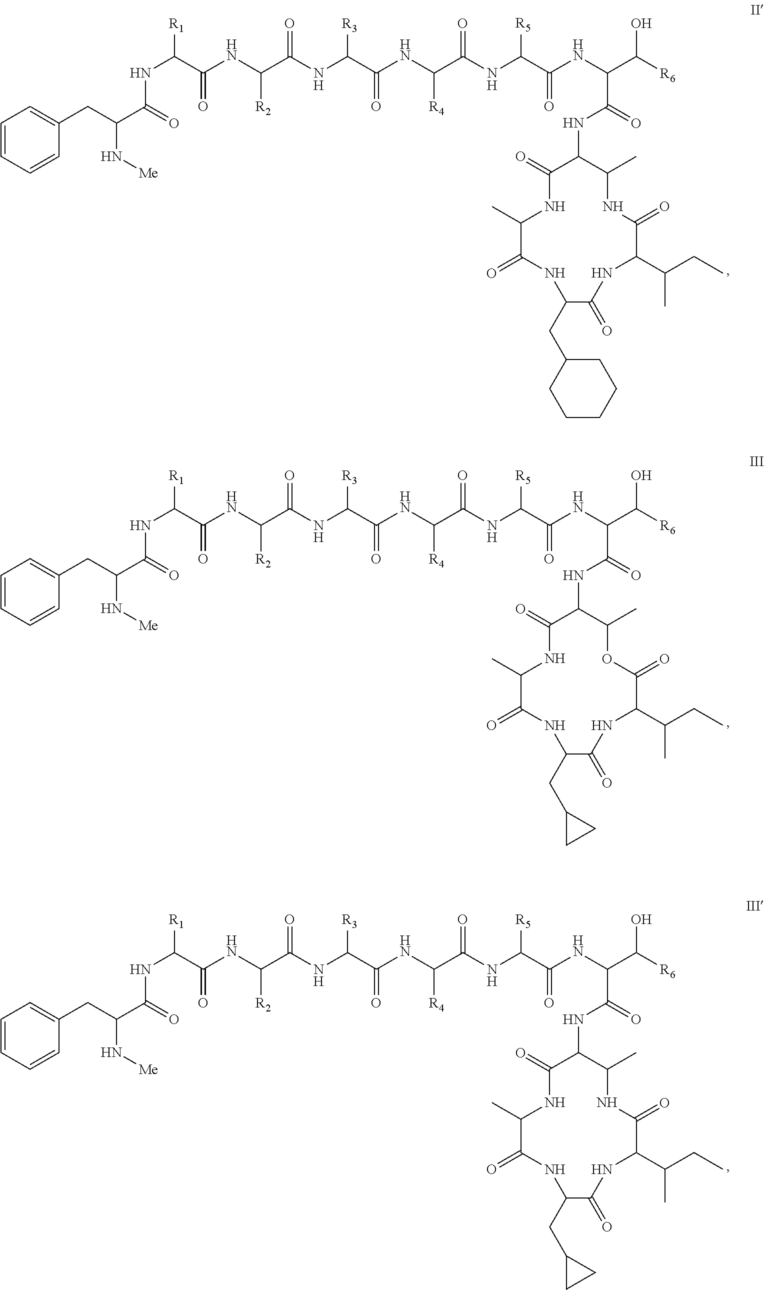

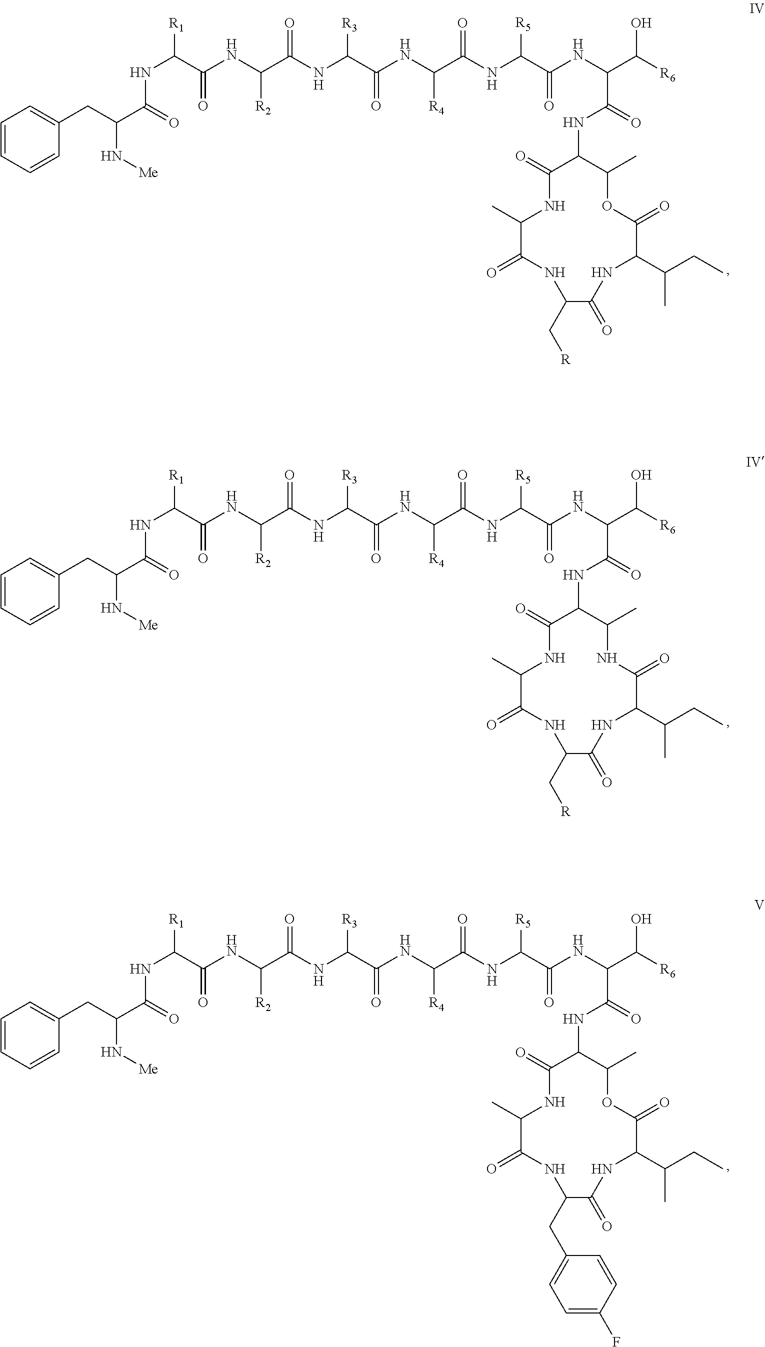

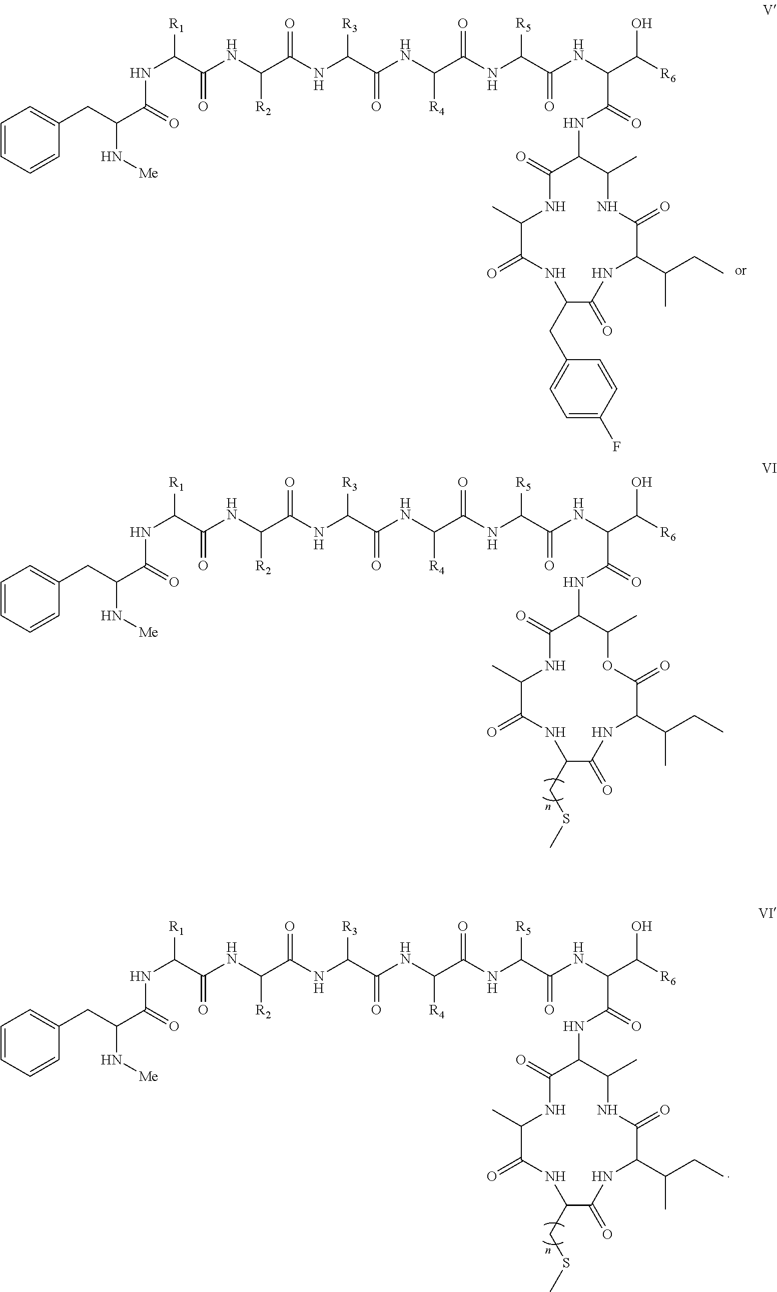

2. The antibiotic of claim 1, wherein each of R₁ to R₅ is a side chain of an amino acid.

3. The antibiotic of claim 1, wherein each of R₁ to R₅ is independently a side chain of leucine, isoleucine, glutamine, serine, threonine, D-allo isoleucine, lysine or arginine.

4. The antibiotic of claim 1, wherein R₁ is the side chain of isoleucine.

5. The antibiotic of claim 1, wherein R₂ is the side chain of serine or threonine.

6. The antibiotic of claim 1, wherein R₃ is the side chain of glutamine.

7. The antibiotic of claim 1 wherein R₄ is the side chain of isoleucine or D-allo isoleucine.

8. The antibiotic of claim 1 wherein R₅ is the side chain of isoleucine or D-allo isoleucine.

9. The antibiotic of claim 1, wherein R₆ is H or a C₁ to C₉ linear or branched alkyl group.

10. The antibiotic of claim 1, wherein R₆ is methyl.

11. The antibiotic of claim 1, wherein R in Formula IV and IV′ is C₂-C₈ linear or branched alkyl chain.

12. The antibiotic of claim 1, wherein R in Formula V and V′ is C₂-C₈ linear or branched alkyl chain.

13. The antibiotic of claim 1, wherein R in Formula VI and VI′ is C₂-C₈ linear or branched alkyl chain.

14. The antibiotic of claim 1, wherein the antibiotic is selected from the antibiotics provided in Table 1.

15. A composition comprising an antibiotic or a salt thereof according to claim 1 and a pharmaceutically acceptable carrier or excipient.

16. A method of treating an infection caused by an agent in a subject, comprising administering to the subject a therapeutically effective amount of an antibiotic according to claim 1.

17. The method of claim 16, wherein the agent is a bacterium, virus, protozoan, helminth, parasite, or fungus.

18. The method of claim 17, wherein the bacterium is one or more of:
Helicobacter pylori, Legionella pneumophilia, Mycobacterium tuberculosis, Mycobacterium avium, Mycobacterium intracellulare, Mycobacterium kansaii, Mycobacterium gordonae, Mycobacteria sporozoites, Staphylococcus aureus, Staphylococcus epidermidis, Neisseria gonorrhoeae, Neisseria meningitidis, Listeria monocytogenes, Streptococcus pyogenes (Group A Streptococcus), Streptococcus agalactiae pyogenes (Group B Streptococcus), Streptococcus dysgalactia, Streptococcus faecalis, Streptococcus bovis, Streptococcus pneumoniae, pathogenic Campylobacter sporozoites, Enterococcus sporozoites, Haemophilus influenzae, Pseudomonas aeruginosa, Bacillus anthracis, Bacillus subtilis, Escherichia coli, Corynebacterium diphtherias, Corynebacterium jeikeium, Corynebacterium sporozoites, Erysipelothrix rhusiopathiae, Clostridium perfringens, Clostridium tetani, Clostridium difficile, Enterobacter aerogenes, Klebsiella pneumoniae, P asturella multocida, Bacteroides thetaiotamicron, Bacteroides uniformis, Bacteroides vulgatus, Fusobacterium nucleatum, Streptobacillus moniliformis, Leptospira, and Actinomyces israelli.

19. The method of claim 17, wherein virus is: Retroviridae, Picornaviridae, Calciviridae, Togaviridae, Flaviridae, Coronaviridae, Rhabdoviridae, Filoviridae, Paramyxoviridae, Orthomyxoviridae, Bungaviridae, Arenaviridae, Reoviridae, Birnaviridae, Hepadnaviridae, Parvoviridae, Papovaviridae, Adenoviridae, Herpesviridae, Poxviridae, and Iridoviridae.

20. The method of claim 17, wherein the protozoan is: Trichomonas vaginalis, Giardia lamblia, Entamoeba histolytica, Balantidium coli, Cryptosporidium parvum, Isospora belli, Trypansoma cruzi, Trypanosoma gambiense, Leishmania donovani, or Naegleria fowleri.

21. The method of claim 17, wherein the helminth is: Schistosoma mansoni, Schistosoma cercariae, Schistosoma japonicum, Schistosoma mekongi, Schistosoma hematobium, Ascaris lumbricoides, Strongyloides stercoralis, Echinococcus granulosus, Echinococcus multilocularis, Angiostrongylus cantonensis, Angiostrongylus constaricensis, Fasciolopis buski, Capillaria philippinensis, Paragonimus westermani, Ancylostoma dudodenale, Necator americanus, Trichinella spiralis, Wuchereria bancrofti, Brugia malayi, and Brugia timori, Toxocara canis, Toxocara cati, Toxocara vitulorum, Caenorhabiditis elegans, or Anisakis spp.

22. The method of claim 17, wherein the parasite is: Plasmodium falciparum, Plasmodium yoelli, Hymenolepis nana, Clonorchis sinensis, Loa boa, Paragonimus westermani, Fasciola hepatica, or Toxoplasma gondii.

23. The method of claim 17, wherein the fungus is: Cryptococcus neoformans, Histoplasma capsulatum, Coccidioides immitis, Blastomyces dermatitidis, Chlamydia trachomatis, Candida albicans, Candida tropicalis, Candida glabrata, Candida krusei, Candida parapsilosis, Candida dubliniensis, Candida lusitaniae, Epidermophyton floccosum, Microsporum audouinii, Microsporum canis, Microsporum canis var. distortum Microsporum cookei, Microsporum equinum, Microsporum ferrugineum, Microsporum falvum, Microsporum gallinae, Microsporum gypseum, Microsporum nanum, Microsporum persicolor, Trichophyton ajelloi, Trichophyton concentricum, Trichophyton equinum, Trichophyton flavescens, Trichophyton gloriae, Trichophyton megnini, Trichophyton mentagrophytes var. erinacei, Trichophyton mentagrophytes var. interdigitale, Trichophyton phaseoliforme, Trichophyton rubrum, Trichophyton rubrum downy strain, Trichophyton rubrum granular strain, Trichophyton schoenleinii, Trichophyton simii, Trichophyton soudanense, Trichophyton terrestre, Trichophyton tonsurans, Trichophyton vanbreuseghemii, Trichophyton verrucosum, Trichophyton violaceum, Trichophyton yaoundei, Aspergillus fumigatus, Aspergillus flavus, or Aspergillus clavatus.

24. A method of killing or inhibiting the growth of an infectious agent, comprising contacting the infectious agent with an effective amount of an antibiotic according to claim 1.
